SEQUENCE LISTING (13 sequences)

SEQ ID NO: 1 - protein, 448 aa, Artificial Sequence
  Eculizumab heavy chain
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial Sequence [sequence omitted]

SEQ ID NO: 3 - protein, 451 aa, Artificial Sequence
  Crovalimab heavy chain
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 4 - protein, 217 aa, Artificial Sequence
  Crovalimab light chain
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 448 aa, Artificial Sequence
  Ravulizumab heavy chain
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 6 - protein, 214 aa, Artificial Sequence [sequence omitted]

SEQ ID NO: 7 - protein, 123 aa, Artificial Sequence [sequence omitted]

SEQ ID NO: 8 - protein, 328 aa, Artificial Sequence
  An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 9 - protein, 110 aa, Artificial Sequence [sequence omitted]

SEQ ID NO: 10 - protein, 107 aa, Artificial Sequence [sequence omitted]

SEQ ID NO: 11 - protein, 650 aa, Artificial Sequence
  Immunoglobulin, anti-(human complement C5 ?-chain); heavy chain; CAS Registry Number 219685-50-4
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGERPAQGGRVSAGSQAQPSCLDAPRLCSPSPGQQGRPHLSPHPEASARPTHAQGEGLLAFSTRLQAGTGWVPLPQALHTQGQVLGSDLPKAISGRTLPPDLSRPQGQTVHSLSSDTFLSSQIRVTPNLLSAERKCCVECPPCPGKPAQASPSSSRRDRCPRVACIQGQPQLGADTSTSISSSAPPVAGPSVFLFPPKPKDTLMISRTPEVTCVVVDVSQEDPEVQFNWYVDGVEVHNAKTKPREEQFNSTDRVVSVLTVLHQDWLNGKEYKCKVSTKASRPPSRKPSPKPKVGPTGCEGHMDRGQLGPPSALGVTAVPTSVPTGQPREPQVYTLPPSQEEMTKNQVSLTCLVKGLYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 214 aa, Artificial Sequence
  Immunoglobulin, anti-(human complement C5 ?-chain); light chain; CAS Registry Number 219685-50-4
DIQMTQSPSSLSASVGDRVTITCGASENIYGALNWYQRKPGKAPKLLIYGATNLADGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

(19) United States
(12) Patent Application Publication
(10) Pub. No.: US 2022/0275070 A1
(43) Pub. Date: Sep. 1, 2022

(54) DOSAGE AND ADMINISTRATION REGIMEN FOR THE TREATMENT OR PREVENTION OF C5-RELATED DISEASES BY THE USE OF THE ANTI-C5 ANTIBODY CROVALIMAB

(71) Applicant: HOFFMANN-LA ROCHE INC., Little Falls, NJ (US)

(72) Inventors: Alexandre Antoine Bernard Sostelly, Basel (CH); Simon Bertrand Marie Buatois, Basel (CH); Antoine Soubret, Basel (CH); Félix Grégoire Jason Jaminion, Basel (CH); Gregor Jordan, Penzberg (DE); Christoph Bucher, Basel (CH); Jean-Eric Charoin, Basel (CH)

(73) Assignee: HOFFMANN-LA ROCHE INC., Little Falls, NJ (US)

(21) Appl. No.: 17/630,046

(22) PCT Filed: Jul. 30, 2020
(86) PCT No.: PCT/EP2020/071555

§ 371 Date: Jan. 25, 2022

(30) Foreign Application Priority Data
Jul. 31, 2019 (EP) 19189442.7
May 14, 2020 (EP) 20174790.4
Jun. 11, 2020 (EP) 20179591.1

(51) Int. Cl. C07K 16/18; A61P 7/00

(52) U.S. Cl. CPC C07K 16/18; A61P 7/00; A61K 2039/505

(57) ABSTRACT

The present invention relates to a dosage and administration regimen of anti-C5 antibodies, particularly of the anti-C5 antibody Crovalimab, for use in a method of treating or preventing C5-related disease in a subject, including paroxysmal nocturnal hemoglobinuria (PNH). The dosage and treatment regimen of the present invention include the administration of an anti-C5 antibody, preferably of the anti-C5 antibody Crovalimab, with loading doses followed by the administration of (a) maintenance dose(s) of the anti-C5 antibody to the subject, wherein the initial administered loading dose is intravenously given to the subject and the remaining loading and maintenance doses are subcutaneously administered in a lower dosage as the intravenously administered loading dose.

The present invention relates to a dosage and administration regimen of anti-C5 antibodies, particularly of the anti-C5 antibody Crovalimab, for use in a method of treating or preventing C5-related disease in a subject, including paroxysmal nocturnal hemoglobinuria (PNH). The dosage and treatment regimen of the present invention include the administration of an anti-C5 antibody, preferably of the anti-C5 antibody Crovalimab, with loading doses followed by the administration of (a) maintenance dose(s) of the anti-C5 antibody to the subject, wherein the initial administered loading dose is intravenously given to the subject and the remaining loading and maintenance doses are subcutaneously administered in a lower dosage as the intravenously administered loading dose.

### BACKGROUND OF THE INVENTION

The complement system plays a central role in the clearance of immune complexes and in immune responses to infectious agents, foreign antigens, virus-infected cells and tumour cells. There are about 25-30 complement proteins, which are found as a complex collection of plasma proteins and membrane cofactors. Complement components achieve their immune defensive functions by interacting in a series of intricate enzymatic cleavages and membrane binding events. The resulting complement cascades lead to the production of products with opsonic, immunoregulatory, and lytic functions.

The complement system can be activated through three distinct pathways: the classical pathway, the lectin pathway, and the alternative pathway. These pathways share many components, and while they differ in their initial steps, they converge and share the same terminal complement components (C5 through C9) responsible for the activation and destruction of target cells.

The classical pathway is normally activated by the formation of antigen-antibody complexes. Independently, the first step in activation of the lectin pathway is the binding of specific lectins such as mannan-binding lectin (MBL), H-ficolin, M-ficolin, L-ficolin and C-type lectin CL-11. In contrast, the alternative pathway spontaneously undergoes a low level of turnover activation, which can be readily amplified on foreign or other abnormal surfaces (bacteria, yeast, virally infected cells, or damaged tissue). These pathways converge at a point where complement component C3 is cleaved by an active protease to yield C3a and C3b.

C3a is an anaphylatoxin. C3b binds to bacterial and other cells, as well as to certain viruses and immune complexes, and tags them for removal from the circulation (the role known as opsonin). C3b also forms a complex with other components to form C5 convertase, which cleaves C5 into C5a and C5b.

C5 is a 190 kDa protein found in normal serum at approximately 80 μg/ml (0.4 μM). C5 is glycosylated with about 1.5-3.0% of its mass attributed to carbohydrate. Mature C5 is a heterodimer of 115 kDa alpha chain that is disulfide linked to 75 kDa beta chain. C5 is synthesized as a single chain precursor protein (pro-C5 precursor) of 1676 amino acids (see, e.g., U.S. Pat. Nos. 6,355,245 B1 and 7,432,356 B1). The pro-C5 precursor is cleaved to yield the beta chain as an amino terminal fragment and the alpha chain as a carboxyl terminal fragment. The alpha chain and the beta chain polypeptide fragments are connected to each other via a disulfide bond and constitute the mature C5 protein.

The terminal pathway of the complement system begins with the capture and cleavage of C5. Mature C5 is cleaved into the C5a and C5b fragments during activation of the complement pathways. C5a is cleaved from the alpha chain of C5 by C5 convertase as an amino terminal fragment comprising the first 74 amino acids of the alpha chain. The remaining portion of mature C5 is fragment C5b, which contains the rest of the alpha chain disulfide bonded to the beta chain. Approximately 20% of the 11 kDa mass of C5a is attributed to carbohydrate.

C5a is another anaphylatoxin. C5b combines with C6, C7, C8 and C9 to form the membrane attack complex (MAC, C5b-9, terminal complement complex (TCC)) at the surface of the target cell. When sufficient numbers of MACs are inserted into target cell membranes, MAC pores are formed to mediate rapid osmotic lysis of the target cells.

As mentioned above, C3a and C5a are anaphylatoxins. They can trigger mast cell degranulation, which releases histamine and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract granulocytes such as neutrophils, eosinophils, basophils and monocytes to the site of complement activation.

The activity of C5a is regulated by the plasma enzyme carboxypeptidase N that removes the carboxy-terminal arginine from C5a forming C5a-des-Arg derivative. C5a-des-Arg exhibits only 1% of the anaphylactic activity and polymorphonuclear chemotactic activity of unmodified C5a.

While a properly functioning complement system provides a robust defense against infecting microbes, inappropriate regulation or activation of complement has been implicated in the pathogenesis of a variety of disorders including, e.g., paroxysmal nocturnal hemoglobinuria (PNH); rheumatoid arthritis (RA); lupus nephritis; ischemia-reperfusion injury; atypical hemolytic uremic syndrome (aHUS); dense deposit disease (DDD); macular degeneration (e.g., age-related macular degeneration (AMD)); hemolysis, elevated liver enzymes, and low platelets (HELLP) syndrome; thrombotic thrombocytopenic purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis (see, e.g., Holers et al., Immunol. Rev. (2008), Vol. 223, pp. 300-316). Therefore, inhibition of excessive or uncontrolled activations of the complement cascade can provide clinical benefits to patients with such disorders.

Paroxysmal nocturnal hemoglobinuria (PNH) is an uncommon blood disorder, wherein red blood cells (erythrocytes) are compromised and are thus destroyed more rapidly than normal red blood cells. PNH results from the clonal expansion of hematopoietic stem cells with somatic mutations in the PIG-A (phosphatidylinositol glycan class A) gene which is located on the X chromosome. Mutations in PIG-A lead to an early block in the synthesis of glycosylphosphatidylinositol (GPI), a molecule which is required for the anchor of many proteins to cell surfaces. Consequently, PNH blood cells are deficient in GPI-anchored proteins, which include complement-regulatory proteins CD55 and CD59. Under normal circumstances, these complement-regulatory proteins block the formation of MAC on cell surfaces, thereby preventing erythrocyte lysis. The absence of the GPI-anchored proteins causes complement-mediated hemolysis in PNH.

PNH is characterized by hemolytic anemia (a decreased number of red blood cells), hemoglobinuria (the presence of hemoglobin in urine, particularly evident after sleeping), and hemoglobinemia (the presence of hemoglobin in the bloodstream). PNH-afflicted subjects are known to have paroxysms, which are defined here as incidences of dark-colored urine. Hemolytic anemia is due to intravascular destruction of red blood cells by complement components. Other known symptoms include dysphasia, fatigue, erectile dysfunction, thrombosis and recurrent abdominal pain.

Eculizumab is a humanized monoclonal antibody directed against the complement protein C5, and the first therapy approved for the treatment of paroxysmal nocturnal hemoglobinuria (PNH) and atypical hemolytic uremic syndrome (aHUS) (see, e.g., Dmytrijuk et al., The Oncologist (2008), 13(9), pp. 993-1000). Eculizumab inhibits the cleavage of C5 into C5a and C5b by C5 convertase, which prevents the generation of the terminal complement complex C5b-9. Both C5a and C5b-9 cause the terminal complement-mediated events that are characteristic of PNH and aHUS (see, e.g., WO-A2 2005/074607, WO-A1 2007/106585, WO-A2 2008/069889, and WO-A2 2010/054403). For the treatment of PNH, the anti-C5 antibodies Eculizumab or Ravulizumab represent the common therapy. However, up to 3.5% of individuals of Asian descent carry polymorphisms in C5 affecting Arg885, which corresponds to the Eculizumab and Ravulizumab binding site (Nishimura et al., N Engl J Med, Vol. 370, pp. 632-639 (2014); DOI: 10.1056/NEJMoa1311084). PNH patients with these polymorphisms experience poor control of intravascular hemolysis with Eculizumab or Ravulizumab, thus constituting a group with a high unmet medical need.

Several reports have described anti-C5 antibodies. For example, WO 95/29697 described an anti-C5 antibody which binds to the alpha chain of C5 but does not bind to C5a, and blocks the activation of C5. WO-A2 2002/30985 described an anti-C5 monoclonal antibody which inhibits C5a formation. On the other hand, WO-A1 2004/007553 described an anti-C5 antibody which recognizes the proteolytic site for C5 convertase on the alpha chain of C5 and inhibits the conversion of C5 to C5a and C5b. WO-A1 2010/015608 described an anti-C5 antibody which has an affinity constant of at least 1×10⁷ M⁻¹. Further, WO-A1 2017/123636 and WO-A1 2017/132259 describe anti-C5 antibodies. Moreover, WO-A 2016/098356 disclosed the generation of an anti-C5 antibody characterized by binding to an epitope within the beta chain of C5 with a higher affinity at neutral pH than at acidic pH. One of the anti-C5 antibodies disclosed in WO-A1 2016/098356 refers to the anti-C5 antibody Crovalimab (see Example 1 below for details). Crovalimab is an anti-C5 antibody that binds to a distinct epitope on the beta subunit of C5, that is different from the Eculizumab/Ravulizumab binding epitope. In vitro studies have demonstrated that the anti-C5 antibody Crovalimab equally binds and inhibits the activity of wild-type and Arg885-mutant C5 (Fukuzawa et al., Sci Rep, 7(1): 1080. doi: 10.1038/s41598-017-01087-7 (2017)). In contrast, WO-A1 2017/104779 reports in FIG. 21 that the anti-C5 antibody Eculizumab did not inhibit the Arg855-mutant C5. Further, WO-A1 2018/143266 relates to pharmaceutical compositions for use in the treatment or prevention of a C5-related disease. Further, WO-A1 2018/143266 discloses dosages and administration schemes of the anti-C5 antibody Crovalimab as used in the COMPOSER study (BP39144). The COMPOSER study refers to a phase I/II global, multicentre, open-label study to assess the safety and efficacy, pharmacokinetics (PK) and pharmacodynamics (PD) of the anti-C5 antibody Crovalimab in healthy subjects and in subjected with PNH. The COMPOSER study contained three parts: Part 1 in healthy participants, Part 2 and Part 3 in patients with paroxysmal nocturnal hemoglobinuria (PNH). Additionally, the patients encompassed in Part 3 of the study were patients who had been treated with the anti-C5 antibody eculizumab for at least 3 months. The participants of Part 1 of the COMPOSER study was designed to include three groups of healthy patients: According to the original protocol design, the first group is a group of patients to whom the anti-C5 antibody Crovalimab is administered intravenously (IV) once at the dose of 75 mg/body; the second group of patients is a group of participants to whom the anti-C5 antibody Crovalimab is administered intravenously (IV) once at the dose of 150 mg/body, and the third group is a group of subjects to whom the anti-C5 antibody crovalimab is administered subcutaneously (SC) once at the dose of 170 mg/body. As Part 1 of the COMPOSER study is adaptive in nature (based on ongoing assessment of safety, tolerability, pharmacokinetics (PK), and pharmacodynamics (pD) data), the actual doses given for Part 1 were: 75 mg IV for the first group of patients, 125 mg IV for the second group of patients, and 100 mg SC for the third group of patients enrolled in Part 1 of the COMPOSER study.

Part 2 of the COMPOSER study was designed to include a group of subjects to whom the anti-C5 antibody Crovalimab is intravenously administered three times: According to the original protocol design, the anti-C5 antibody Crovalimab was initially administered at a dose of 300 mg/body (IV), then at 500 mg/body (IV) a week after the initial administration, and finally at 1000 mg/body (IV) two weeks after the second administration. Starting from two weeks after the final intravenous administration, the anti-C5 antibody crovalimab is administered subcutaneously once a week at the dose of 170 mg/body. Based on the emerging clinical data from Part 1 and the PK simulation, the starting dose for patients in Part 2 of the COMPOSER study has been changed from 300 mg to 375 mg IV. Thus, the actual doses given in Part 2 of the COMPOSER study are as follows: The anti-C5 antibody Crovalimab is initially administered intravenously (IV) at a dose of 375 mg/body, followed by a dose of 500 mg/body (IV) a week after the initial administration, and finally at 1000 mg/body (IV) two weeks after the second administration. Starting from two weeks after the final intravenous administration, the anti-C5 antibody Crovalimab is administered subcutaneously (SC) once a week at the dose of 170 mg/body.

Part 3 of the study included patients which were treated with the anti-C5 antibody Eculizumab for three months preceding enrolment in the trial and the patients had to receive regular infusions of Eculizumab. Part 3 of the study was designed to include three groups of subjects. The anti-C5 antibody Crovalimab is initially administered to the subjects of all groups intravenously once at the dose of 1000 mg/body. Starting from one week after the initial intravenous administration (day 8 after the initial IV administration), the anti-C5 antibody Crovalimab is subcutaneously administered to subjects of the first group once every week at the dose of 170 mg/body, to subjects of the second group once every two weeks at the dose of 340 mg/body, and to subjects of the third group once every four weeks at the dose of 680 mg/body. In COMPOSER Part 3, Drug-Target-Drug-Complexes (DTDCs) between Crovalimab, human C5 and the antibody Eculizumab were detected in all patients with PNH who switched from the anti-C5 antibody Eculizumab to Crovalimab. DTDCs trigger transient increase of Crovalimab clearance that can potentially increase the risk of temporary loss of complete inhibition of the terminal complement pathway (see Roth et al., Blood (2020), Vol. 135, pp. 912-920; doi: 10.1182/blood.2019003399 and Sostelly et al., Blood (2019), Vol. 134, p. 3745).

Moreover, WO-A1 2018/143266 describes that immunocomplexes (Drug-Target-Drug-Complexes) between Crovalimab, human C5 and the antibody Eculizumab could be formed in subjects, that have been treated with Eculizumab. When subjects, particularly subjects who need complete C5 inhibition maintained, such as PNH or aHUS patients, switch from the anti-C5 antibody Eculizumab to Crovalimab, both anti-C5 antibodies are present in blood circulation and form Drug-Target-Drug-Complexes (DTDCs) since they bind to different epitopes of the human C5. These DTDCs are built from repetition of Eculizumab-C5-Crovalimab-C5 chain of molecules and can grow when two DTDCs assemble to form a larger DTDC. The treatment goal of patients encompassed in Part 3 of the COMPOSER study with Crovalimab is to ensure a rapid and sustained complete inhibition of the terminal complement pathway. However, Drug-Target-Drug-Complexes (DTDCs) consisting of Crovalimab, human C5, and Eculizumab were detected in all patients switching from Eculizumab in COMPOSER Part 3. DTDCs and particularly large DTDCs are cleared more slowly and are more likely to cause toxicity. As the formation of such DTDCs may cause potential risks, such as circulatory impairment, vasculitis risk due to the complex sizes, type III hypersensitivity reactions, or abnormal activation of the complement system, the formation of such DTDCs should be avoided (see also Roth et al., Blood (2020), Vol., 135, pp. 912-920; doi: 10.1182/blood.2019003399).

Further, based on its mechanism of action, the anti-C5 antibody Crovalimab inhibits complement-mediated lysis of red-blood cells (erythrocytes) lacking complement regulatory proteins. If the terminal complement pathway is temporarily not blocked during the treatment interval, these red-blood cells (erythrocytes) will be lysed, and it may lead to breakthrough hemolysis, which is a severe clinical complication in PNH patients. Biological stress (infection, surgery, pregnancy) leads to a physiological activation of the complement pathway with upregulation of C5 (Schutte et al., Int Arch Allergy Appl Immunol. (1975), Vol. 48(5), pp. 706-720). In patients with PNH, it is therefore important to not only maintain complete blockade of the terminal complement activity throughout the dosing interval, but to also maintain a reserve of Crovalimab free binding sites to minimize the occurrence of breakthrough hemolysis.

Accordingly, there is a need to identify a dosing and administration regimen that (1) minimizes the formation of DTDCs in patients suffering from C5-related diseases, and particularly in patients switching from the anti-C5 antibody Eculizumab to Crovalimab, (2) maximizes the level of Crovalimab free binding sites, and (3) ensures that patients remain above an anti-C5 antibody target threshold concentration required for terminal complement inhibition despite the inter-individual variability.

### SUMMARY OF THE INVENTION

This need is addressed by the present invention by providing the embodiments as defined in the claims.

The present invention relates to an anti-C5 antibody for use in a method of treating or preventing a C5-related disease in a subject, wherein the method comprises the consecutive steps of:
(a) intravenously administering a loading dose of 1500 mg of the anti-C5 antibody to the subject once, followed by subcutaneously administering at least one loading dose of 340 mg of the anti-C5 antibody to the subject; and
(b) subcutaneously administering at least one maintenance dose of 1020 mg of the anti-C5 antibody to the subject.

In the context of the present invention, the subject to be treated is preferably a patient with a body weight of equal or greater than 100 kg. In the context of the present invention the subject to be treated is/are subject/s which suffer from a C5-related disease which require complement activity inhibition (for example PNH and aHUS). Moreover, the invention is directed to the use of the anti-C5 antibody for the treatment or prevention of a C5-related disease, particularly PNH. In the context of the present invention, the present invention is directed to the treatment or prevention of a C5-related disease, preferably PNH, in patients that has been treated with one pharmaceutical product useful for the treatment or prevention of the C5-related disease, preferably PNH, and wherein the intravenously administered loading dose of the anti-C5 antibody is administered to the subject after the final dose of the pharmacological product. Accordingly, the herein described dosage and administration regimen of the anti-C5 antibody, particularly of the anti-C5 antibody Crovalimab, is given to patients who has been treated with one pharmaceutical product useful for the treatment or prevention of the 05-related disease, preferably PNH. As explained in more detail below, the pharmaceutical product useful for the treatment of the C5-related disease which has been given to the subjects before the start of the claimed dosage and treatment regimen refers to the anti-C5 antibody Eculizumab or Ravulizumab, preferably to the anti-C5 antibody Eculizumab.

As shown in the appended Examples, the dose and treatment regimen as defined in the claims ensure a sustained and consistent blockade of terminal complement activity (with approximately more than 95% of subjects being maintained above the target threshold of 100 μg/ml); see FIGS. 4 and 7. Further, a terminal complement inhibition was achieved immediately following the initial dose and generally maintained throughout dosing interval; see FIG. 8. Further, the dosage and treatment regimen of the present invention also ensure sufficient reserve of free binding sites for the majority of the dosing interval in both treatment-naïve and Eculizumab pre-treated patients; see FIG. 2. Crovalimab and Eculizumab bind to different C5 epitopes and thus DTDCs are expected to be formed. DTDCs are expected to develop if patients are exposed to Crovalimab and Eculizumab simultaneously (see FIG. 5), during a switch period from Eculizumab to the anti-C5 antibody Crovalimab. The formation of DTDCs may contribute to increase Crovalimab clearance and may cause potential risks such as type III hypersensitivity reactions as explained above. In patients switching from Eculizumab to Crovalimab, the dose and treatment regimen as defined in the claims is expected to reduce the formation of DTDCs; see FIGS. 3 and 12. Accordingly, the herein described dosage and treatment regimen outlines a novel and improved dosage regimen of anti-C5 antibodies, preferably of the anti-C5 antibody Crovalimab for the treatment or prevention of a C5-related disease, preferably PNH. The safety and therapeutic efficacy of the claimed dosage and treatment regimen is further reported in FIGS. 9 to 11.

Accordingly, the present invention relates to an anti-C5 antibody, preferably the anti-C5 antibody Crovalimab, for use in a method of treating or preventing a C5-related disease in a subject, preferably a subject with a body weight of equal or greater than 100 kg, wherein the method comprises the consecutive steps of:
(a) intravenously administering a loading dose of 1500 mg of the anti-C5 antibody to the subject once, followed by subcutaneously administering at least one loading dose of 340 mg of the anti-C5 antibody to the subject; and
(b) subcutaneously administering at least one maintenance dose of 1020 mg of the anti-C5 antibody to the subject.

The “loading dose” refers to the dose of the anti-C5 antibody administered to the subject suffering from a C5-related disease, preferably PNH, at the beginning of the treatment, i.e. at the start of the treatment regimen. In pharmacokinetics (PK), a “loading dose” is an initial higher dose of a drug that may be given to a patient at the beginning of a course of treatment before dropping down to a lower dose. In the context of the present invention, the loading dose is firstly given to subjects to be treated by intravenous administration, followed by subcutaneous administration. In the context of the present invention, the loading dose is given once at a dose of 1500 mg. Accordingly, in the context of the present invention, a loading dose of a composition formulated for intravenous administration is given intravenously once to the subject before one loading dose or more loading doses of a pharmaceutical composition formulated for subcutaneous administration is/are given subcutaneously.

In the context of the present invention, a loading dose or more loading doses of the anti-C5 antibody is/are subcutaneously administered to the patients after the intravenous administration of a loading dose of 1500 mg of the anti-C5 antibody. The subcutaneously administered loading dose(s) is (are) subcutaneously administered at a dose of 340 mg of the anti-C5 antibody at least once to the subject 1 day to 3 weeks (21 days) after the start of the intravenous administration of the anti-C5 antibody. Accordingly, in the context of the present invention, a loading dose of 340 mg of the anti-C5 antibody is subcutaneously administered at least once to the subject 1, 2, 3, 4, 5, 6, 7, 8, 9, 10, 11, 12, 13, 14, 15, 16, 17, 18, 19, 20, or 21 days after the start of the intravenous administration of the anti-C5 antibody. Preferably, a loading dose of 340 mg of the anti-C5 antibody is administered to the subject 1 day after the start of the intravenous administration of the anti-C5 antibody. More preferably, one loading dose of 340 mg of the anti-C5 antibody is subcutaneously administered 1 day after the start of the intravenous administration. In the context of the present invention, at least one additional loading dose of 340 mg of the anti-C5 antibody is subcutaneously administered to the subject 1 week (7 days), 2 weeks (14 days), or 3 weeks (21 days) after the start of the intravenous administration of the anti-C5 antibody. Most preferably, additional loading doses of 340 mg of the anti-C5 antibody are subcutaneously administered 1 week (7 days), 2 weeks (14 days) and 3 weeks (21 days) after the start of the intravenous administration of the anti-C5 antibody. Accordingly, within the context of the present invention 1, 2, 3, 4 and/or 5 loading doses is/are given to the subject, wherein one loading dose, preferably the initial loading dose is intravenously administered at a dose of 1500 mg to the subject, and wherein 1, 2, 3 or 4 loading doses is/are given subcutaneously at a dose of 340 mg to the patient. In the context of the present invention, the subcutaneous administration of 4 loading doses each having a dosage of 340 mg of the anti-C5 antibody is preferred, wherein the additional loading doses are subcutaneously administered once 1 day after the start of the intravenous administration of the anti-C5 antibody, followed by subcutaneous administration of loading doses 1 week, 2 weeks and 3 weeks once weekly after the start of the intravenous administration of the anti-C5 antibody. Accordingly, a total amount of 2860 mg of an anti-C5 antibody may be administered to the patient with loading doses. The total amount refers to the total doses of the anti-C5 antibody administered after 22 days of the treatment, i.e. the dose reached at the end of day 22 of the treatment that is calculated by adding the loading doses at days 1 (the loading dose of 1500 mg initially administered intravenously), 2 (first subcutaneously administered loading dose of 340 mg given to the patient 1 day after the start of the intravenous administration of the anti-C5 antibody), 8 (second subcutaneously administered loading dose of 340 mg given 1 week after the start of the intravenous administration), 15 (third subcutaneously administered loading dose of 340 mg given 2 weeks after the start of the intravenous administration), and 22 (fourth subcutaneously administered loading dose of 340 mg given 3 weeks after the start of the intravenous administration). For example, the total amount of the anti-C5 antibody given via (a) loading dose(s) corresponding to an intravenous administration of 1500 mg (day 1), followed by subcutaneous administration of 340 mg (day 2), 340 mg (day 8), 340 mg (day 15) and 340 mg (day 22) is 2860 mg.

According to the present invention, the initial dose or doses is/are followed by subsequent doses of equal or smaller amounts of anti-C5 antibody at intervals sufficiently close to maintain the concentration of the anti-C5 antibody at or above an efficacious target level. Accordingly, in the context of the present invention, (a) maintenance dose(s) is (are) administered to the patients after the loading dose(s). The “maintenance dose” refers to the dose of the anti-C5 antibody that is given to a subject suffering from a C5-related disease to maintain the concentration of the anti-C5 antibody above a certain efficacious threshold of the anti-C5 antibody concentration. In the context of the present invention the target level of the anti-C5 antibody is approximately 100 μg/ml or more. The target level of the anti-C5 concentration within the present invention may be determined in a biological sample of the subject to be treated. Means and methods for the determination of the anti-C5 concentration in a biological sample are within the common knowledge of the skilled person and can for example be determined by an immunoassay. Preferably in the context of the present invention, the immunoassay is an ELISA. Likewise, the hemolytic activity can be used as a parameter for the efficacious treatment of patients suffering from a C5-related disease by the claimed dosage and treatment regimen. In the context of the hemolytic activity can be determined in a biological sample of the patient to be treated. In the context of the present invention the complete terminal complement inhibition (complete inhibition of the terminal pathway of the complement system) can be defined by a hemolytic activity which is less than 10 U/mL It is preferred that the hemolytic activity is less than 10 U/mL, i.e. 10, 9, 8, 7, 6, 5, 4, 3, 2, 1, or 0 U/mL. Means and method for the determination of the hemolytic activity in a biological sample of patients to be treated by the dosage and administration regimen according to the invention are known by the skilled person. Exemplarily, the hemolytic activity can be determined by an immunoassay. Preferably in the context of the present invention, the immunoassay is an ex vivo liposome immunoassay (LIA). In the context of the present invention, the biological sample is a blood sample. Preferably, the blood sample is a red-blood sample (erythrocytes). Preferably, the maintenance dose(s) is (are) subcutaneously administered to the patients, at a dose or doses of 1020 mg of the anti-C5 antibody. Accordingly, within the context of the present invention at least one maintenance, or more maintenance doses is/are given to the subject, wherein the maintenance dose(s) is (are) subcutaneously administered at a dose of 1020 mg. In the context of the present invention, at least one maintenance dose of 1020 mg of the anti-C5 antibody is subcutaneously administered to the subject 4 weeks (28 days) after the start of the intravenous administration of the anti-C5 antibody. Preferably, a maintenance dose of 1020 mg is subcutaneously administered to the subjects once 4 weeks after the start of the intravenous administration of the anti-C5 antibody. Accordingly, within the context of the present invention at least one maintenance dose of 1020 mg is subcutaneously administered to the patient, 4 weeks (28 days) after the start of the intravenous administration of the anti-C5 antibody, i.e. on day 29 of the treatment regimen. Accordingly, in the context of the present invention, the maintenance dose of 1020 mg is subcutaneously administered, preferably once 4 weeks (28 days) after the start of the intravenous administration of the anti-C5 antibody. In the context of the present invention, a total amount of 3880 mg of an anti-C5 antibody may be administered to the patient with loading doses and the maintenance dose in accordance with the present invention. The total amount refers to the total doses of the anti-C5 antibody administered after 29 days of the treatment, i.e. the dose reached at the end of day 29 of the treatment that is calculated by adding the loading doses at days 1 (the loading dose of 1500 mg initially administered intravenously), 2 (first subcutaneously administered loading dose of 340 mg given to the patient 1 day after the start of the intravenous administration of the anti-C5 antibody), 8 (second subcutaneously administered loading dose of 340 mg given 1 week after the start of the intravenous administration), 15 (third subcutaneously administered loading dose of 340 mg given 2 weeks after the start of the intravenous administration), 22 (fourth subcutaneously administered loading dose of 340 mg given 3 weeks after the start of the intravenous administration), and the subcutaneously administered maintenance dose of 1020 mg (day 29). For example, the total amount of the anti-C5 antibody given via the loading dose and the maintenance dose corresponding to an intravenous administration of 1500 mg (day 1), followed by subcutaneous administration of 340 mg (day 2), 340 mg (day 8), 340 mg (day 15), 340 mg (day 22) and 1020 mg (day 29) is 3880 mg.

The subcutaneous administration of a maintenance dose of 1020 mg can be repeated several times with time intervals of 4 weeks (Q4W). It is preferred in the context of the present invention that maintenance dose of 1020 mg is repeated at least 1, 2, 3, 4, 5, 6, 7, 8, 9, 10, 11, 12, 24, 36, 48 months. Preferred in the context of the present invention is the repetition of the maintenance dose of 1020 mg with time intervals of 4 weeks and continues for the patient's whole life.

In particular, the present invention relates to an anti-C5 antibody for use in a method of treating or preventing a C5-related disease in a subject, preferably in a subject with a body weight of equal or greater than 100 kg, wherein the method comprises the consecutive steps of:
(i) intravenously administering a loading dose of 1500 mg of the anti-C5 antibody to the subject once;
(ii) subcutaneously administering a loading dose of 340 mg of the anti-C5 antibody to the subject 1 day after the start of the intravenous administration of the anti-C5 antibody;
(iii) subcutaneously administering a loading dose of 340 mg of the anti-C5 antibody to the subject 1 week (7 days), 2 weeks (14 days) and 3 weeks (21 days) after the start of the intravenous administration of the anti-C5 antibody once weekly;
(iv) subcutaneously administering a maintenance of 1020 mg of the anti-C5 antibody to the subject 4 weeks (28 days) after the start of the intravenous administration of the anti-C5 antibody; and
(v) repeating step (iv) several times with time intervals of 4 weeks (28 days).

The terms “intravenous administration”/“intravenously administering” refer in the context of the present invention to the administration of the anti-C5 antibody into a vein of the subject such that the body of the patient to be treated receives the anti-C5 antibody in approximately 15 minutes or less, preferably 5 minutes or less. For intravenous administration, the anti-C5 antibody has to be formulated that it be administered via a suitable device such as (but not limited to) a syringe. In the context of the present invention, the formulation for intravenous administration comprises 50 to 350 mg of the anti-C5 antibody, 1 to 100 mM of a buffering agent, such as histidine/aspartic acid comprising a pH of 5.5±1.0, 1 to 100 mM of an amino acid such as arginine, and 0.01 to 0.1% of a non-ionic surfactant, such as a poloxamer. Preferred in the context of the present invention, the formulation for intravenous administration is provided in a 2 mL glass vial containing the following components: 170 mg/ml Crovalimab, 30 mM histidine/aspartic acid (pH 5.8), 100 mM arginine hydrochloride and 0.05% Poloxamer 188™. The formulation is then administered to the patient within a tolerated time period, such as 5 minutes, 15 minutes, 30 minutes, 90 minutes, or less. Moreover, the formulation for intravenous administration is given to the patients to be treated with an injection volume of between 1 ml to 15 ml, preferably about 9 ml.

The terms “subcutaneous administration”/“subcutaneously administering” refer in the context of the present invention to the introduction of the anti-C5 antibody under the skin of an animal or human patient, preferable within a pocket between the skin and underlying tissue, by relatively slow, sustained delivery from a drug receptacle. The pocket may be created by pinching or drawing the skin up and away from underlying tissue. For subcutaneous administration, the anti-C5 antibody has to be formulated that it may be administered via a suitable device such as (but not limited to) a syringe, a prefilled syringe, an injection device, an infusion pump, an injector pen, a needless device, or via a subcutaneous patch delivery system. In the context of the present invention, the formulation for subcutaneous administration comprises 50 to 350 mg of the anti-C5 antibody, 1 to 100 mM of a buffering agent, such as histidine/aspartic acid comprising a pH of 5.5±1.0, 1 to 100 mM of an amino acid such as arginine, and 0.01 to 0.1% of a non-ionic surfactant, such as a poloxamer. Preferred in the context of the present invention, the formulation for intravenous administration is provided in a 2.25 prefilled syringe containing the following components: 170 mg/ml Crovalimab, 30 mM histidine/aspartic acid (pH 5.8), 100 mM arginine hydrochloride and 0.05% Poloxamer 188™. In the context of the present invention a formulation for the subcutaneous administration is provided in a prefilled syringe with a needle safety device. The injection devices for subcutaneous administration comprises about 1 to 15 ml or more, preferably 2.25 ml of a formulation for subcutaneous administration comprising the anti-C5 antibody. Under normal circumstances, the injection volume to be subcutaneously administered is 1 to 15 ml, preferably either 2 ml (340 mg Crovalimab), or 6 ml (1020 mg Crovalimab). In the context of the present invention, the subcutaneous administration refers to introduction of the anti-C5 antibody under the skin of the patient to be treated by relatively slow, sustained delivery from a drug receptacle for a period of time including, but not limited to, 30 minutes or less, 90 minutes or less. Optionally, the administration may be made by subcutaneous implantation of a drug delivery pump implanted under the skin of the patient to be treated, wherein the pump delivers a predetermined amount of the anti-C5 antibody for a predetermined period of time, such as 30 minutes, 90 minutes, or a time period spanning the length of the treatment regimen.

In the context of the present invention the above dosages and treatment regimens can be useful for the treatment or prevention of a C5-related disease in a subject who has been treated with at least one pharmacological product for use in treatment or prevention of the disease once or more times. For example, the treatment regimen of the present invention can be useful for treating a patient having a C5-related disease who has received prior treatment with at least one pharmacological product for use in a method of treating or preventing the disease but is expected to better respond to the treatment regimen according to the present invention. In such cases, the medication can be switched from the pharmacological product to the anti-C5 antibodies for use in the treatment or prevention of a C5-related disease in accordance with the present invention. Preferably, the intravenously administered loading dose of the anti-C5 antibody is given to the subject to be treated after the final dose of the pharmaceutical product. The intravenously administered loading dose of the anti-C5 antibody has preferably a dose of 1500 mg.

In the context of the present invention, the pharmacological product comprises an active substance which is different from the anti-C5 antibody which is given in accordance to the present invention either intravenously or subcutaneously. The active substance of pharmacological product can in the context of the present invention be an siRNA targeting C5 mRNA, or an anti-C5 antibody which is different from the anti-C5 antibody subcutaneously or intravenously administered to the subject to be treated in accordance with the present invention. The pharmacological product may comprise an anti-C5 antibody which is different antibody from the anti-C5 antibody given to the patients in the context of the present invention. The antibody comprised in the pharmaceutical product that has been used in the prior treatment may be Ravulizumab, or Eculizumab or variants thereof. Preferably, the antibody comprised in the pharmacological product that has been used in the prior treatment is Eculizumab or its variants. Exemplarily sequence variants of the anti-C5 antibody Eculizumab are shown in SEQ ID NOs: 11 and 12.

Antibody variants in the context of the present invention may be anti-C5 antibodies that comprise an Fc region variant in which one or more amino acid modifications have been introduced into a native sequence Fc region of an antibody. The Fc region variant may comprise a human Fc region sequence (e.g., a human IgG1, IgG2, IgG3 or IgG4 Fc region) comprising an amino acid modification (e.g., a substitution) at one or more amino acid positions. In the context of the present invention, an antibody variant possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc gamma R binding (hence likely lacking ADCC activity), but retains FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 B1 (see, e.g., Hellstrom et al., Proc. Nat'l Acad. Sci. USA (1983), Vol. 83, pp. 7059-7063) and Hellstrom et al., Proc. Nat'l Acad. Sci. USA (1985), Vol. 82, pp. 1499-1502; U.S. Pat. No. 5,821,337 B1 (see Bruggemann et al., J. Exp. Med. (1987), Vol. 166, pp. 1351-1361). Alternatively, non-radioactive assays methods may be employed (see, for example, ACTI™ non-radioactive cytotoxicity assay for flow cytometry (CellTechnology, Inc. Mountain View, Calif.); and CytoTox 96 (registered trademark) non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA (1998), Vol. 95, pp. 652-656. C1q binding assays may also be carried out to confirm that the antibody is unable to bind C1q and hence lacks CDC activity. See, e.g., C1q and C3c binding ELISA in WO-A2 2006/029879 and WO-A1 2005/100402. To assess complement activation, a CDC assay may be performed (see, for example, Gazzano-Santoro et al., J. Immunol. Methods (1996), Vol. 202, pp. 163; Cragg et al., Blood (2003), Vol. 101, pp. 1045-1052 and Cragg et al., Blood (2004), Vol. 103, pp. 2738-2743). FcRn binding and in vivo clearance/half-life determinations can also be performed using methods known in the art (see, e.g., Petkova et al., Intl. Immunol. (2006), Vol. 18(12), pp. 1759-1769).

Antibodies with reduced effector function include those with substitution of one or more of Fc region residues 238, 265, 269, 270, 297, 327 and 329 (U.S. Pat. No. 6,737,056 B1). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581 B1).

Certain antibody variants with improved or diminished binding to FcRs are described. (See, e.g., U.S. Pat. No. 6,737,056 B1; WO-A2 2004/056312, and Shields et al., J. Biol. Chem. (2001), Vol. 9(2), pp. 6591-6604).

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551 B1, WO 1999/51642, and Idusogie et al., J. Immunol. (2000), Vol. 164, pp. 4178-4184.

Antibodies with increased half-lives and improved binding to the neonatal Fc receptor (FcRn), which is responsible for the transfer of maternal IgGs to the fetus (Guyer et al., J. Immunol. (1976), Vol. 117, pp. 587 and Kim et al., J. Immunol. (1994), Vol. 24, pp. 249) are described in US 2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826 B1).

See also Duncan, Nature (1988), Vol. 322, pp. 738-740, U.S. Pat. No. 5,648,260 B1; U.S. Ser. No. 15/624,821 and WO 1994/29351 concerning other examples of Fc region variants.

In the context of the present invention the initial dose of the composition for intravenous injection in the present invention is administered on the same day as, or 1 day, 2 days, 3 days, 4, days, 5 days, 6 days, 7 days (1 week), 8 days, 9 days, 10 days, 11 days, 12 days, 13 days, 14 days (2 weeks), 15 days, 16 days, 17 days, 18 days, 19 days, 20 days, 21 days (3 weeks), or more days after the final dose of the pharmacological product is administered to the patient to be treated. Preferably, in the context of the present invention, the intravenously administered loading dose of the anti-C5 antibody is administered on the 3 day, or after 3 days, 4, days, 5 days, 6 days, 7 days (1 week), 8 days, 9 days, 10 days, 11 days, 12 days, 13 days, 14 days (2 weeks), 15 days, 16 days, 17 days, 18 days, 19 days, 20 days, 21 days (3 weeks), or more days after the final dose of the pharmacological product. Preferably, the intravenously administered loading dose of the anti-C5 antibody is given to the patient 7 days (1 week), or more days after the final dose of the pharmacological product. Also preferred in the context of the present invention is the intravenous administration of the loading dose 14 days (2 weeks), or more days after the final dose of the pharmacological product. Most preferred in the context of the present invention, is the intravenous administration of the anti-C5 antibody 21 days (3 weeks) after the final dose of the pharmacological product.

In the context of the present invention, a “week” refers to a period of time of 7 days.

In the context of the present invention, a “month” refers to a period of time of 4 weeks.

“Treatment” in the context of the present invention comprises the sequential succession of an “induction treatment” and at least a “maintenance treatment”. Typically, a treatment according to the invention comprises an “induction treatment” and at least one “maintenance treatment”. Typically, a treatment according to the invention may be 1 month, 2 months, 3 months, 4 months, 5 months, 6 months, 7 months, 8 months, 9 months, 10 months, 11 months, 1 year (12 months), 2 years (24 months), 3 years (36 months), or 4 years (48 months). Preferred in the context of the present invention is a treatment that continues for the patient's whole life.

An “induction treatment” consists in the sequential succession of (i) an intravenous administration of a loading dose, preferably a dose of 1500 mg, of the anti-C5 antibody to the subject, and (ii) a subcutaneous administration of at least one loading dose, preferably a dose of 340 mg, of the anti-C5 antibody to the subject. As explained herein above, it is preferred within the context of the present invention that a loading dose of 340 mg of the anti-C5 antibody is given 1 day, 1 week (7 days), 2 weeks (14 days) and 3 weeks (21 days) after the intravenously administered loading dose was given to the subject. Preferably, the loading dose to be administered intravenously has a dose of 1500 mg. The loading dose which is subcutaneously given to the subject to be treated has a dose of 1360 mg. Thus, in the context of the present invention a loading dose of 2860 mg is either intravenously, or subcutaneously administered to the subject to be treated during the induction treatment. A “maintenance treatment” consists in the sequential succession of (i) a maintenance period wherein one or more maintenance dose(s) is (are) subcutaneously given to the subjects. In the context of the present invention, it is preferred that a maintenance dose of 1020 mg of the anti-C5 antibody is given to the subject, preferably once, 4 weeks (1 month) after the start of the intravenous administration of the loading dose of the anti-C5 antibody. As explained above, the subcutaneous administration of a maintenance dose of 1020 mg can be repeated several times with time intervals of 4 weeks (Q4W). Preferred in the context of the present invention is the repetition of the maintenance dose of 1020 mg with time intervals of 4 weeks and continues for the patient's whole life.

In the context of the present invention, the C5-related disease is a complement-mediated disease or condition which involves excessive or uncontrolled activation of C5. In certain embodiments, the C5-related disease is at least one selected from a group consisting of paroxysmal nocturnal hemoglobinuria (PNH), rheumatoid arthritis (RA), lupus nephritis, ischemia-reperfusion injury, atypical hemolytic uremic syndrome (aHUS), dense deposit disease (DDD), macular degeneration, hemolysis, elevated liver enzymes, low platelets (HELLP) syndrome, thrombotic thrombocytopenic purpura (TTP), spontaneous fetal loss, Pauci-immune vasculitis, epidermolysis bullosa, recurrent fetal loss, multiple sclerosis (MS), traumatic brain injury, an injury resulting from myocardial infarction, cardiopulmonary bypass or hemodialysis, refractory generalized myasthenia gravis (gMG), and neuromyelitis optica (NMO). Preferably, in the context of the present invention the C5-related disease is at least one selected from a group consisting of PNH, aHUS, gMG and NMO. Most preferably, the C5-related disease is PNH. Further, in the context of the present invention the subject suffering from the C5-related disease PNH may be tested for the presence of the Arg885-mutation of C5. Accordingly, the herein disclosed dosage regimen may also be used for the treatment and/or prevention of subjects suffering from PNH characterised in that the subjects have the Arg855-mutation of C5. In the context, Arg885-mutation means a genetic variation of C5 where Arg at position 885 is substituted by His. In this context, the term “C5” refers to a protein having the amino acid sequence as shown in SEQ ID NO: 13.

In the context of the present invention, the anti-C5 antibody is preferably Crovalimab. The sequence details of the anti-C5 antibody Crovalimab (CAS number: 1917321-26-6) are disclosed in List No. 119 of proposed International Non-proprietary Names for Pharmaceutical Substances (INN) as published at pages 302 and 303 of WHO Drug Information (2018), Vol. 32, No. 2. The sequences of the anti-C5 antibody Crovalimab is also shown in SEQ ID NO: 3 (heavy chain) and SEQ ID NO: 4 (light chain). The generation of the anti-C5 antibody Crovalimab used in the present invention is described in WO 2016/098356 (see Example 1 for details). Further, in the context of the present invention, the anti-C5 antibody Crovalimab is administered to the patients by a formulation either for intravenous administration, or for subcutaneous administration. Preferred in the context of the present invention is the intravenous or subcutaneous administration of the herein provided dosages as (a) fixed-dose(s).

The formulation for intravenous administration comprises 50 to 350 mg of the anti-C5 antibody Crovalimab, 1 to 100 mM of a buffering agent, such as histidine/aspartic acid comprising a pH of 5.5±1.0, 1 to 100 mM of an amino acid such as arginine, and 0.01 to 0.1% of a non-ionic surfactant, such as a poloxamer. Preferred in the context of the present invention, the formulation for intravenous administration is provided in a 2 mL glass vial containing the following components: 170 mg/ml Crovalimab, 30 mM histidine/aspartic acid (pH 5.8), 100 mM arginine hydrochloride and 0.05% Poloxamer 188™.

The formulation for subcutaneous administration comprises 50 to 350 mg of the anti-C5 antibody Crovalimab, 1 to 100 mM of a buffering agent, such as histidine/aspartic acid comprising a pH of 5.5±1.0, 1 to 100 mM of an amino acid such as arginine, and 0.01 to 0.1% of a non-ionic surfactant, such as a poloxamer. Preferred in the context of the present invention, the formulation for intravenous administration is provided in a 2.25 prefilled syringe containing the following components: 170 mg/ml Crovalimab, 30 mM histidine/aspartic acid (pH 5.8), 100 mM arginine hydrochloride and 0.05% Poloxamer 188™.

The anti-C5 antibody Eculizumab is sold under the trade name Solids® by the company Alexion Pharmaceuticals, Inc. The sequences of the anti-C5 antibody Eculizumab are shown in SEQ ID NO: 1 (heavy chain) and SEQ ID NO: 2 (light chain). Further, sequence variants of the anti-C5 antibody Eculizumab are shown in SEQ ID NOs: 11 and 12.

The sequences of the anti-C5 antibody Ravulizumab is sold under the trade name Ultomiris® by the company Alexion Pharmaceuticals, Inc. The sequences of the anti-C5 antibody Ravulizumab (CAS number: 1803171-55-2) are disclosed in List No. 117 of proposed International Non-proprietary Names for Pharmaceutical Substances (INN) as published at pages 319 and 320 of WHO Drug Information (2017), Vol. 31, No. 2. The sequences of the anti-C5 antibody Ravulizumab are also shown in SEQ ID NO: 5 (heavy chain) and SEQ ID NO: 6 (light chain).

Patients described in the context of the present invention are patients suffering from a C5-related disease. Preferred patients in the context of the present invention are patients with a body weight of equal or greater than 100 kg. In the context of the present invention, the C5-related disease is a complement-mediated disease or condition which involves excessive or uncontrolled activation of C5. In certain embodiments, the C5-related disease is at least one selected from a group consisting of paroxysmal nocturnal hemoglobinuria (PNH), rheumatoid arthritis (RA), lupus nephritis, ischemia-reperfusion injury, atypical hemolytic uremic syndrome (aHUS), dense deposit disease (DDD), macular degeneration, hemolysis, elevated liver enzymes, low platelets (HELLP) syndrome, thrombotic thrombocytopenic purpura (TTP), spontaneous fetal loss, Pauci-immune vasculitis, epidermolysis bullosa, recurrent fetal loss, multiple sclerosis (MS), traumatic brain injury, an injury resulting from myocardial infarction, cardiopulmonary bypass or hemodialysis, refractory generalized myasthenia gravis (gMG), and neuromyelitis optica (NMO). Preferably, in the context of the present invention the C5-related disease is at least one selected from a group consisting of PNH, aHUS, gMG and NMO. Most preferably, the C5-related disease is PNH.

Moreover, the present invention relates to a method of treating or preventing a C5-related disease in a subject, wherein the method comprises the consecutive steps of:
(a) intravenously administering a loading dose of 1500 mg of the anti-C5 antibody to the subject once, followed by subcutaneously administering at least one loading dose of 340 mg of the anti-C5 antibody to the subject; and
(b) subcutaneously administering at least one maintenance dose of 1020 mg of the anti-C5 antibody to the subject.

It is preferred in the context of the present invention that the method of treating or preventing a C5-related disease in a subject is carried out by the following administration steps:
(i) intravenously administering a loading dose of 1500 mg of the anti-C5 antibody to the subject once;
(ii) subcutaneously administering a loading dose of 340 mg of the anti-C5 antibody to the subject 1 day after the start of the intravenous administration of the anti-C5 antibody;
(iii) subcutaneously administering a loading dose of 340 mg of the anti-C5 antibody to the subject 1 week, 2 weeks and 3 weeks after the start of the intravenous administration of the anti-C5 antibody once weekly;
(iv) subcutaneously administering a maintenance of 1020 mg of the anti-C5 antibody to the subject 4 weeks after the start of the intravenous administration of the anti-C5 antibody; and
(v) repeating step (iv) several times with time intervals of 4 weeks.

As explained above, it is preferred in the context of the present invention that the anti-C5 antibody used in the context of the dosage and administration regiment is Crovalimab. Further, the definition given above apply likewise to the above methods of treating or preventing a C5-related disease. It is also preferred in the context of the present invention that the subject to be treated has a body weight of equal or greater than 100 kg.

### THE FIGURES SHOW

FIG. 1: Relationship between the anti-C5 antibody Crovalimab and the hemolytic activity as measured by liposome immunoassay (LIA) and healthy subjects and subjects with the C5-related disease paroxysmal nocturnal hemoglobinuria (PNH)

The assessment of the exposure-response relationship demonstrates that approximately 100 μg/mL of Crovalimab is required to achieve complete terminal complement inhibition. The complete terminal complement inhibition (complete inhibition of the terminal pathway of the complement system) is defined as hemolytic activity <10 U/mL. The vertical dotted line marks the pharmacodynamics (PD) threshold of 100 μg/ml Crovalimab.

FIG. 2: Available free binding sites of the anti-C5 antibody Crovalimab

Grey lines correspond to the simulation of 15 individuals based on the parameters estimated from the COMPOSER (BP39144) data. The data of the COMPOSER study were used for the simulations. The y-axis shows the concentration of the anti-C5 antibody Crovalimab (R07112689; SKY59). The x-axis shows the time in days. Dark grey lines correspond to the median values of these 15 patients. S0: COMPOSER Part 3 regimen S5: Proposed regimen in Part 4 of the COMPOSER study and Phase III.

FIG. 3: Time profile of the Drug-Target-Drug-Complex (DTDC)

Grey lines correspond to the simulation of 15 individuals based on the parameters estimated from the COMPOSER (BP39144) data. The data of the COMPOSER study were used for the simulations. Dark grey lines correspond to the median values of these 15 patients. S0: COMPOSER Part 3 regimen; S5: Proposed regimen in Part 4 of the COMPOSER study and Phase III; R07112689: Crovalimab (SKY59).

FIG. 4: Simulated Concentration-Time Profiles of Crovalimab in Treatment Naïve Patients (upper panel) and Patients with PNH Switching Treatment from Eculizumab to Crovalimab (lower panel)

Grey interval corresponds to the 90% prediction interval and the grey line to the predicted median. The black dashed line corresponds to the 100 μg/ml target concentration level of the anti-C5 antibody Crovalimab.

FIG. 5: Model describing how Drug-Target-Drug-Complexes (DTDCs) between Crovalimab, human C5 and the antibody Eculizumab are cleared, recycled and sequentially built from smaller DTDCs

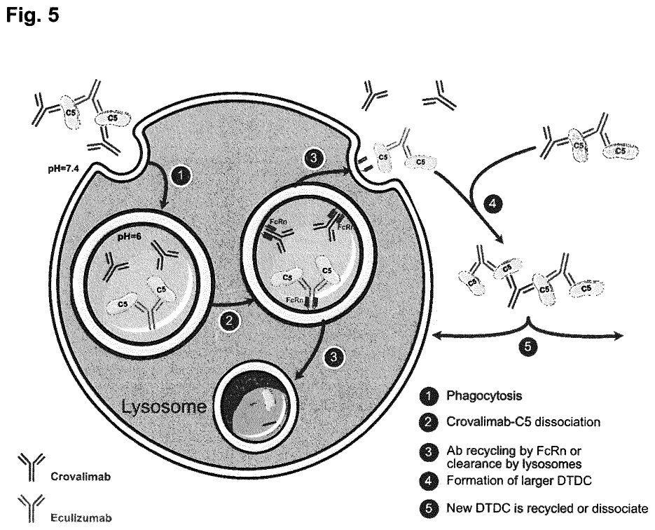

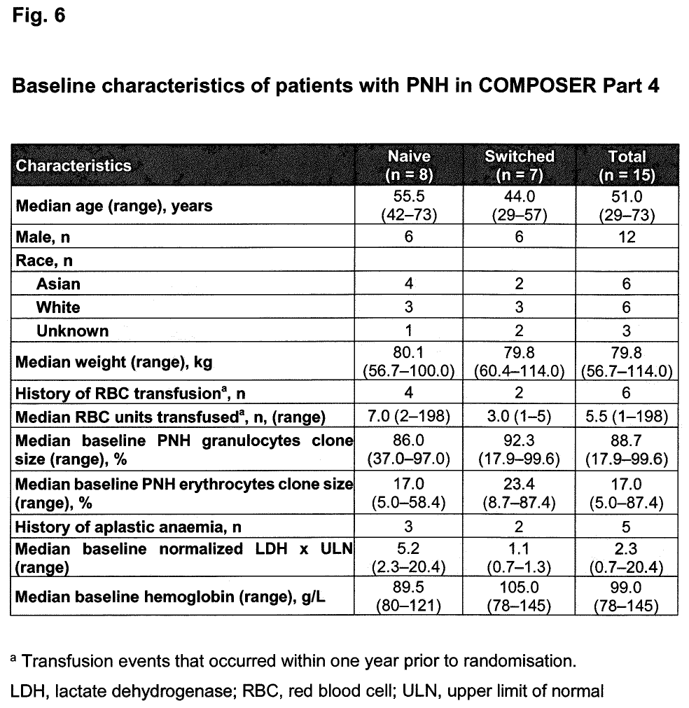

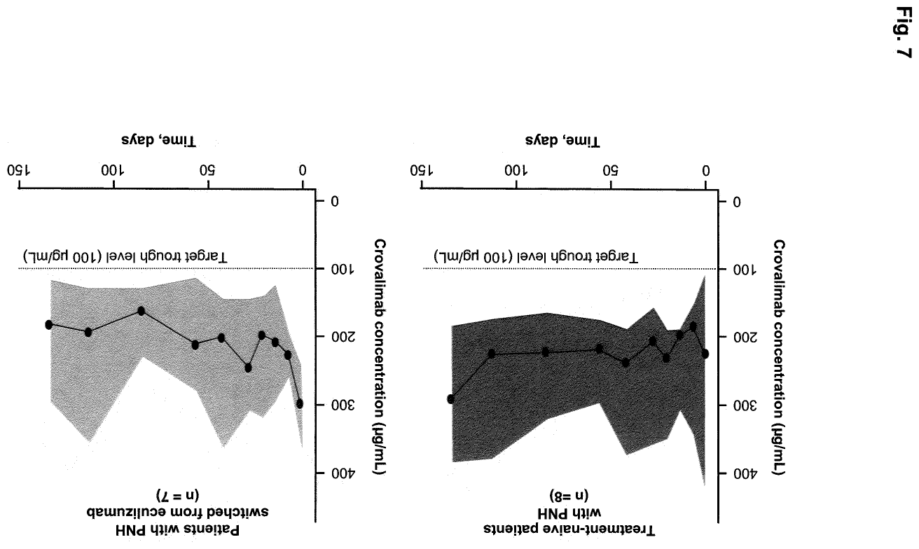

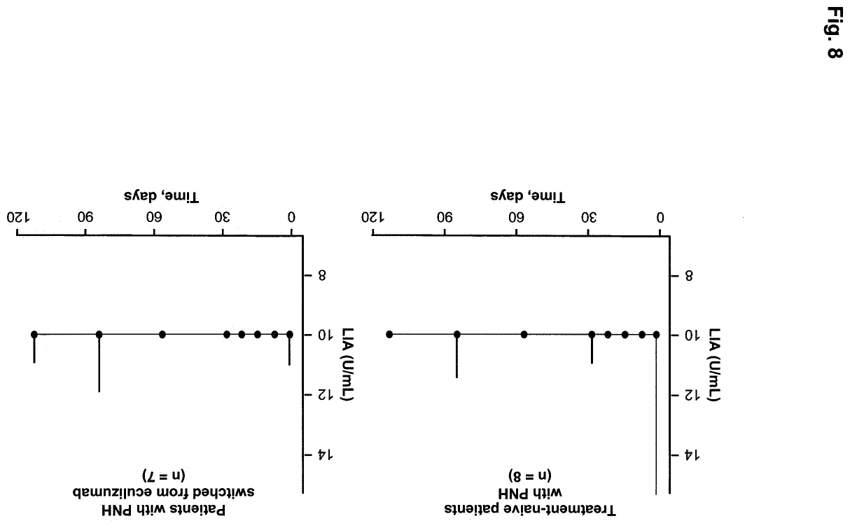

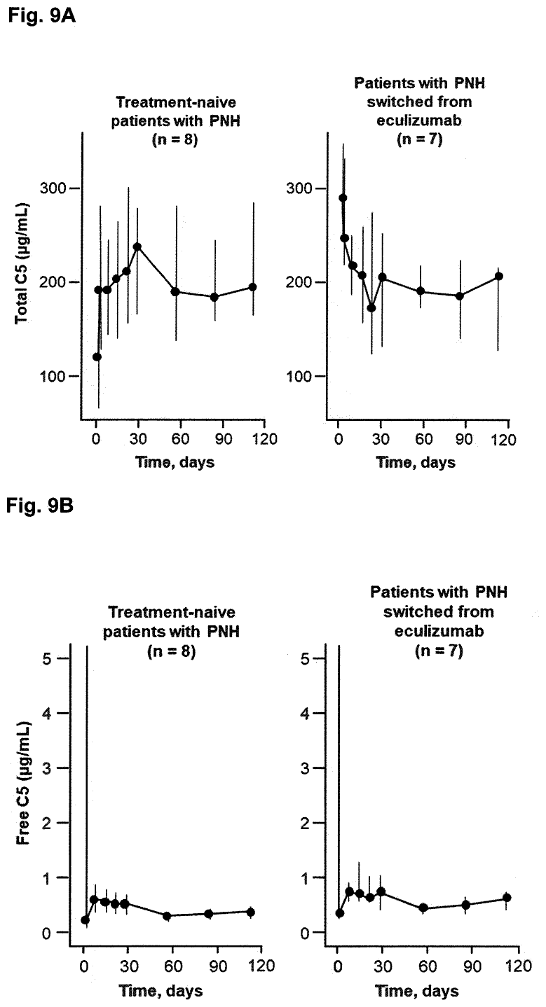

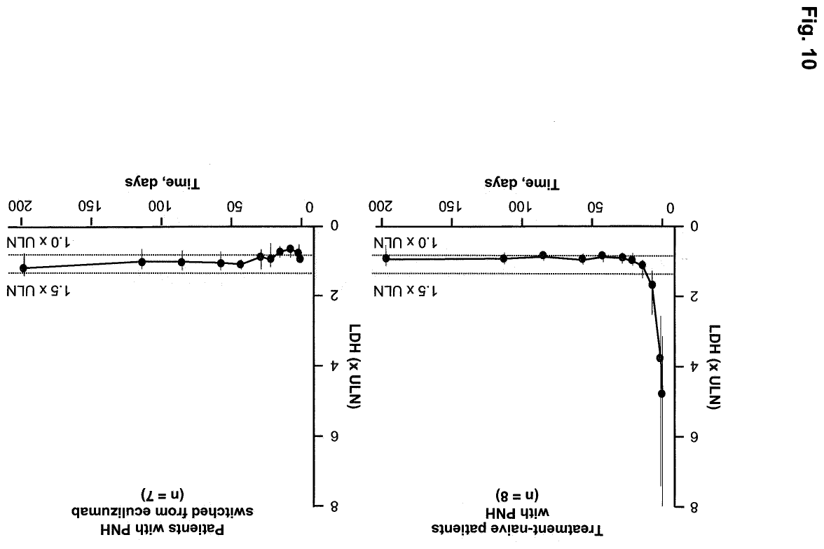

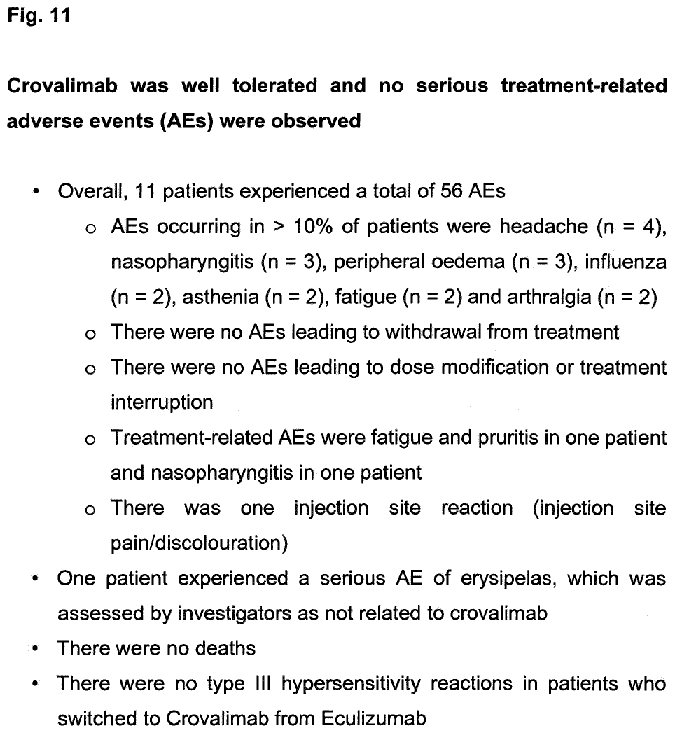

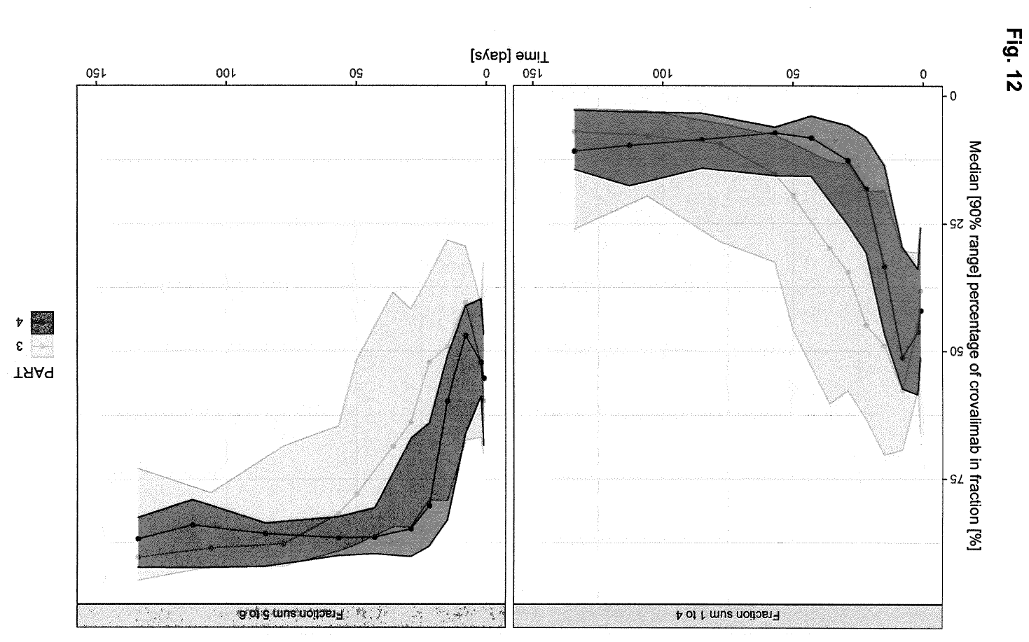

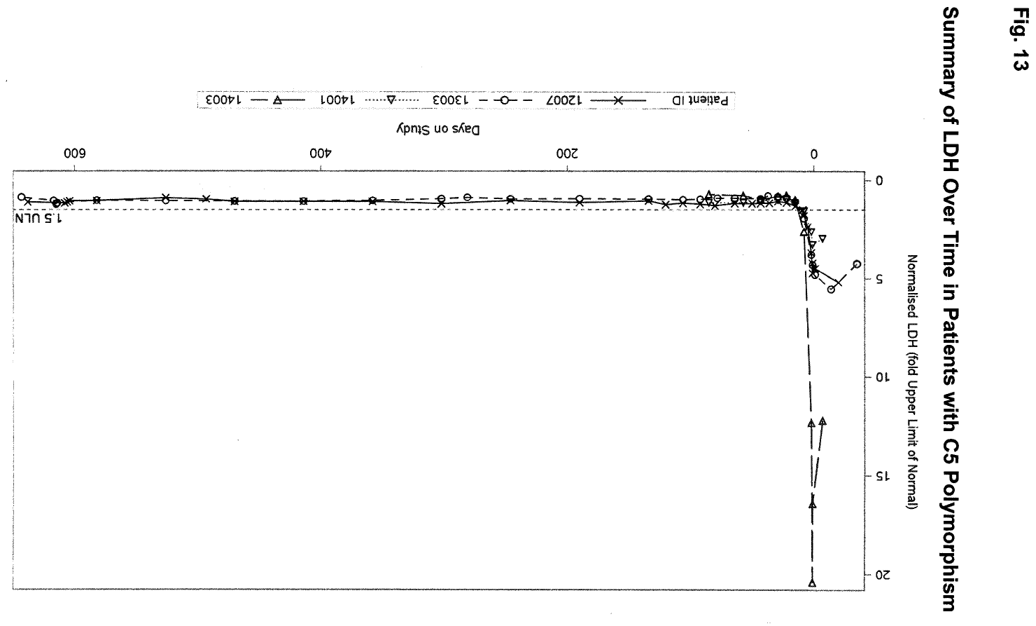

When patients switch from the anti-C5 antibody Eculizumab to Crovalimab, both anti-C5 antibodies are present in blood circulation and form DTDCs since they bind to different epitopes of the human C5. These DTDCs are built from repetition of Eculizumab-C5-Crovalimab-C5 chain of molecules and grow over time when two DTDCs assemble to form a larger DTDC. The model (FIG. 5) reports how DTDCs are cleared and recycled by the FcRn receptors of the anti-C5 antibody Crovalimab. (1) DTDCs are developed if patients are exposed to Crovalimab and Eculizumab simultaneously during a switch period from 1 drug to the other due the differential epitope recognition of C5 by the antibodies. The DTDCs are taken via phagocytosis into endosomes. (2) The Crovalimab antibody which binds to the human C5 in a pH-dependent manner dissociates from the soluble human C5—that has been bound to the anti-C5 antibody Crovalimab—under acidic conditions (pH 6.0) in the endosome, whereas the anti-C5 antibody Eculizumab still binds to the soluble human C5 under the acidic conditions in the endosome. (3) The anti-C5 antibodies (the anti-C5 antibody Crovalimab and the C5-Eculizumab complex) are taken up by the cells by binding to the FcRn expressed on the cell membrane. The C5-Eculizumab complex is translocated to a lysosome to be degraded or recycled with the 05-protein still bound to the antibody. In contrast, the anti-C5 antibody Crovalimab has an improved functionality/efficacy because it dissociates from the FcRn in the endosome under acidic conditions to be released back into the plasma without the C5 protein. (4), (5) The released anti-C5 antibody Crovalimab is available to bind again to human C5 and to build up further, smaller DTDCs. This has the effect of “recycling” the anti-C5 antibody Crovalimab. The DTDCs and particularly the C5-Eculizumab complexes are subsequently again degraded by the endosomes while the anti-C5 antibody Crovalimab is again recycled to build up smaller DTDCs.

FIG. 6: Part 4 of COMPOSER included patients with PNH

COMPOSER Part 4 evaluated the safety, pharmacokinetics (PK), and pharmacodynamics (PD) effects of an optimised crovalimab regimen in patients with PNH who were naïve to anti-C5 therapy, preferably to Crovalimab therapy, or who were switched from Eculizumab, with primary assessment after 20 weeks. Of the 15 enrolled patients, 8 (53%) had not previously received therapy with a C5 inhibitor and 7 (47%) were switched from Eculizumab to Crovalimab.

FIG. 7: Crovalimab exposure in patients enrolled in Part 4 of the COMPOSER study

All patients maintained Crovalimab levels above the C_{trough} value of approximately 100 μg/mL, which is associated with terminal complement activity inhibition. The lines represent the mean value, and shaded area shows the 95% confidence interval.

FIG. 8: Liposome immunoassay (LIA) time course showing median complement activity in the patients enrolled in Part 4 of the COMPOSER study

Terminal complement inhibition was achieved immediately following the initial dose and generally maintained throughout the study period. The lines represent the median value, and the whiskers show the 95% confidence interval. The lower limit of quantification for the LIA assay is 10 U/mL. LIA, liposome immunoassay.

FIG. 9: Measurement of the total and free C5 levels in the patients enrolled in Part 4 of the COMPOSER study

(A) A limited total C5 accumulation was observed in naïve patients, and a decline was seen in switched patients. (B) Free C5 levels declined rapidly following initial dose and remained low throughout the follow-up period.

FIG. 10: Measurement of the normalised lactate dehydrogenase (LDH) level in the patients enrolled in Part 4 of the COMPOSER study

In naive patients, median lactate dehydrogenase (LDH) levels declined to ≤1.5×upper limit of normal (ULN) by day 15 and remained below that level throughout the observation period. In patients who switched from Eculizumab to Crovalimab, median baseline LDH was ≤1.5×ULN and remained so throughout the observation period. LDH, lactate dehydrogenase; ULN, upper limit of normal.

FIG. 11: Summery of the Crovalimab treatment-related adverse events (AEs)

Crovalimab was well tolerated and no serious treatment-related adverse events (AEs) were observed.

FIG. 12: Observed DTDC Profiles Over Time with Part 3 and Part 4 Crovalimab Regimens of the COMPOSER study

Solid lines are the sum of the median percentages of crovalimab eluted in the size exclusion chromatography (SEC) fractions 1 to 4 (left panels) and fractions 5 to 6 (right panels). The dosage regimen of Part 3 of the COMPOSER study is shown in light grey and the dosage regiment of Part 4 is shown in dark grey.

FIG. 13: Normalized LDH levels of PNH patients carrying C5 Arg885His mutation treated with Crovalimab

Crovalimab achieved sustained terminal complement inhibition in PNH patients with Arg885 polymorphism. All patients achieved complete terminal complement inhibition as measured by liposome immunoassay (LIA). LIA levels ranged from 32-42 U/mL at study entry and declined to <10 U/mL by day 2 and were maintained thereafter. The lower limit of quantification for the LIA assay is 10 U/mL. LIA, liposome immunoassay.

### THE FOLLOWING EXAMPLES ILLUSTRATE THE INVENTION

### Example 1: The Anti-C5 Antibodies

The sequences of the anti-C5 antibody Crovalimab are shown in SEQ ID NO: 3 (heavy chain) and SEQ ID NO: 4 (light chain). Further, the generation of the anti-C5 antibody Crovalimab used in the present invention is described in WO 2016/098356. Briefly, the genes encoding the heavy chain variable domain (VH) of 305L015 (SEQ ID NO: 7)) were combined with the genes encoding a modified human IgG1 heavy chain constant domain (CH) variant SG115 (SEQ ID NO: 8). The genes encoding the light chain variable domain (VL) of 305LO15 (SEQ ID NO: 9) were combined with the genes encoding a human light chain constant domain (CL) (SK1, SEQ ID NO: 10). Antibodies were expressed in HEK293 cells co-transfected with the combination of heavy and light chain expression vectors, and were purified by protein.

### Example 2: Dosages and Administration Regimens Used in the COMPOSER Study (BP39144; ClinicalTrials.gov Identifier: NCT03157635)

To determine suitable dosages and administration regimen, the phase I/II COMPOSER study (BP39144) was initiated. The study initially consisted of three parts: Part 1 in healthy participants, Part 2 and Part 3 in patients with paroxysmal nocturnal hemoglobinuria (PNH). Additionally, the patients encompassed in Part 3 of the study were patients who had been treated with the anti-C5 antibody Eculizumab for at least 3 months.

Part 1 of the study was designed to include three groups of healthy patients. The first group is a group of patients to whom the anti-C5 antibody Crovalimab is administered intravenously (IV) once at the dose of 75 mg/body. The second group of patients is a group of participants to whom the anti-C5 antibody Crovalimab is administered intravenously (IV) once at the dose of 150 mg/body. The third group is a group of subjects to whom the anti-C5 antibody Crovalimab is administered subcutaneously (SC) once at the dose of 170 mg/body. As Part 1 of the COMPOSER study is adaptive in nature (based on ongoing assessment of safety, tolerability, pharmacokinetics (PK), and pharmacodynamics (pD) data), the actual doses given for Part 1 were: 75 mg IV for the first group of patients, 125 mg IV for the second group of patients, and 100 mg SC for the third group of patients enrolled in Part 1 of the COMPOSER study.

Part 2 of the study was designed to include a group of subjects to whom the anti-C5 antibody Crovalimab is intravenously administered three times: According to the original protocol design, the anti-C5 antibody Crovalimab was initially administered at a dose of 300 mg/body (IV), then at 500 mg/body (IV) a week after the initial administration, and finally at 1000 mg/body (IV) two weeks after the second administration. Starting from two weeks after the final intravenous administration, the anti-C5 antibody Crovalimab is administered subcutaneously (SC) once a week at the dose of 170 mg/body. Based on the emerging clinical data from Part 1 and the PK simulation, the starting dose for patients in Part 2 of the COMPOSER study has been changed from 300 mg to 375 mg IV. Thus, the actual doses given in Part 2 of the COMPOSER study are as follows: The anti-C5 antibody Crovalimab is initially administered intravenously (IV) at a dose of 375 mg/body, followed by a dose of 500 mg/body (IV) a week after the initial administration, and finally at 1000 mg/body (IV) two weeks after the second administration. Starting from two weeks after the final intravenous administration, the anti-C5 antibody Crovalimab is administered subcutaneously (SC) once a week at the dose of 170 mg/body.

Part 3 of the study included patients which were treated with the anti-C5 antibody Eculizumab for at least three months preceding enrolment in the trial and the patients had to receive regular infusions of Eculizumab. Part 3 of the study was designed to include three groups of subjects. The anti-C5 antibody Crovalimab is initially administered to the subjects of all groups intravenously once at the dose of 1000 mg/body. Starting from one week after the initial intravenous administration (day 8 after the IV administration), the anti-C5 antibody Crovalimab is subcutaneously (SC) administered to subjects of the first group once every week at the dose of 170 mg/body, to subjects of the second group once every two weeks at the dose of 340 mg/body, and to subjects of the third group once every four weeks at the dose of 680 mg/body. 15 healthy patients were enrolled in Part 1 of the COMPOSER study. Part 1 was randomized, so only 9 of the initial 15 patients got Crovalimab. 19 patients were enrolled in Part 3 of the COMPOSER study, but three patients have discontinued.

The details of the patients included by the COMPOSER study (Part 1, Part 2 and Part 3 can be summarized as follows:

| | Mean (SD) | | | |
|---|---|---|---|---|
| | Median (Min/Max) | | | |
| Covariate | All Subjects (n = 35) | Part 1 (n = 9) | Part 2 (n =10) | Part 3 (n = 16) |
| Age (years) | 48 (13) | 37.6 (10.9) | 53.9 (11.8) | 50.3 (11.8) |
| | 47 (24/74) | 36 (24/52) | 52.5 (35/74) | 49 (33/69) |
| Body Mass Index (kg/m²) | 25.3 (6.84) | 22.4 (2.16) | 26 (3.87) | 26.6 (9.36) |
| | 24.4 (15.7/50.1) | 21.6 (19.9/26.2) | 24.6 (21.6/33.4) | 25.5 (15.7/50.1 |
| Body Surface Area (m²) | 1.88 (0.249) | 1.91 (0.157) | 1.86 (0.231) | 1.87 (0.307) |
| | 1.89 (1.38/2.28) | 1.96 (1.65/2.13) | 1.80 (1.56/2.21) | 1.91 (1.38/2.28) |
| Height (cm) | 172.7 (10.2) | 179.8 (7.33) | 169.8 (10.4) | 170.4 (10) |
| | 173 (153/189) | 177 (169/189) | 170 (153/184) | 167.5 (156/189) |
| Body Weight (kg) | 75.6 (20.3) | 72.7 (9.90) | 75.4 (16) | 77.3 (26.9) |
| | 72.3 (40.6/131.5) | 72.8 (56.7/87.8) | 67.7 (58.7/98) | 72.9 (40.6/131.5) |

After generation of the above details of the patients included by Parts 1 to 3 of the COMPOSER study, one additional patient of Part 3 COMPOSER study has discontinued from the study.

### Example 3: Determination of a Dosage Regimen to Achieve Complete and Sustained Terminal Complement Inhibition Throughout the Treatment with the Anti-C5 Antibody Crovalimab

The treatment goal for Crovalimab in C5-related diseases such as preferably paroxysmal nocturnal hemoglobinuria (PNH) is to ensure a rapid and sustained complete inhibition of the terminal complement pathway. In patients switching from Eculizumab to Crovalimab a washout period is clinically inappropriate. Therefore, by design, residual concentrations of Eculizumab are present when Crovalimab dosing is initiated. Drug-Target-Drug-Complexes (DTDCs) consisting of Crovalimab, human C5, and Eculizumab were detected in all patients switching from Eculizumab in COMPOSER Part 3 using a multiplex assay combining size exclusion chromatography (SEC) with an enzyme linked immunosorbent assay (ELISA). SEC is a separation technique based on the difference in the stokes radius and geometry of proteins: SEC separates molecules according to differences in size as they pass through a gel filtration medium packed in a column to form a packed bed. Unlike ion exchange or affinity chromatography, molecules do not bind to the chromatography medium so buffer medium composition does not directly affect resolution (the degree of separation between peaks). The medium is a porous matrix of spherical particles with chemical and physical stability and inertness (lack of reactivity and adsorptive properties). SEC was used in fractionation mode to separate multiple components in a sample on the basis of differences in their size. For complex sample composition with different proteins like serum, combination of SEC with an analyte (Crovalimab)-specific ELISA provided the desired specificity and sensitivity to detect Crovalimab concentrations in each of the separated fractions. To enable the detection of Crovalimab concentrations with the ELISA, the SEC separation is fractionated in eight fractions. For each individual, a DTDC profile over time was described using this approach. To determine the dosing regimen expected to achieve complete and sustained terminal complement inhibition throughout the dosing interval, two complementary model-informed drug development (MIDD) approaches were developed to recommend the dose to be used in the clinical trial (Phase III dose):
An empirical population pharmacokinetics model used to recommend a subcutaneous (SC) dose and regimen maintaining Crovalimab concentrations above a target threshold concentration of 100 μg/ml throughout the dosing interval in the patients.
A biochemical model describing simultaneously the kinetics of total and free C5, the pharmacokinetics of Crovalimab and Eculizumab, and the kinetics of DTDCs used to recommend a dose and regimen minimizing the formation of large DTDCs in patients switching from Eculizumab to Crovalimab and maximizing the level of free Crovalimab binding sites in all the patients.

### 3.1 Population Pharmacokinetics Model

The concentration-time profiles of the anti-C5 antibody Crovalimab were best described using a two-compartment open model with first-order elimination and a first-order absorption to describe the subcutaneous (SC) administration (see Betts A. et al., mAbs (2018), Vol. 10, No. 5, pp. 751-764). Pharmacokinetics (PK) profiles in patients switching treatment from Eculizumab in COMPOSER Part 3 show a transient faster elimination not observed in healthy volunteers and treatment-naïve patients with PNH. To describe the pharmacokinetics (PK) for patients switching treatment from Eculizumab to the anti-C5 antibody Crovalimab, elimination of Crovalimab was modeled as a combination of the first-order elimination used for treatment-naïve patients and a faster clearance, which decreases exponentially across time. Body weight (Median: 72.3 (40.6-131.5) [kg]) was tested as a covariate for the clearances and volumes and was found to significantly influence these parameters when incorporated using allometric scaling with a coefficient fixed to 0.75 for the clearances and 1 for the volumes The parameter “clearance” is the measure of the ability of the body to eliminate a drug. Clearance is expressed as a volume per unit of time. The parameter “volumes” stands for the volume of distribution, a measure of the apparent space in the body available to contain the anti-C5 antibody Crovalimab. Age was also found as a covariate on the absorption rate and was introduced in the model as a categorical covariate. Patients with an age greater or equal to 50 years old appeared to have a lower absorption rate than younger patients. Bioavailability following subcutaneous (SC) administration is estimated to be approximately 100%.

The model was able to precisely estimate the PK parameters and had good predictive performances that qualifies its use for simulation purposes.

### 3.2 Drug-Target-Drug Complexes (DTDC) Biochemical Model

A biochemical mathematical model was developed to investigate the kinetics of DTDCs formation and elimination under the assumption that complexes of increased size are formed by the reversible binding of smaller complexes (see FIG. 5). This model accounts for all complexes made of the Ab1-Ag-Ab2 unit repetition (antibody 1 (Ab1), antibody 2 (Ab2), and antigen (Ag) represent Crovalimab, Eculizumab, and C5, respectively) starting from the smallest complexes (Ab1-Ag-Ab2) up to the largest complexes containing 4 Ab1, 4 Ab2 and 8 Ag (e.g., the complex Ab1-Ag-Ab2-Ag-Ab1-Ag-Ab2-Ag-Ab1-Ag-Ab2-Ag-Ab1-Ag-Ab2-Ag) as observed in the in vitro SEC assays. Each possible biochemical reaction describing the formation of a complex through the binding of 2 smaller complexes were described using a ligand binding model. The clearance of the complexes and the recycling of free Crovalimab from the DTDCs (due to SMART-Ig Recycling® releasing C5 from Crovalimab in acidic condition of the lysosome were also accounted for in each binding reaction. Details of the SMART-Ig Recycling® system was described by Fukuzawa et al., Sci Rep. (2017), Vol. 7(1): 1080; doi: 10.1038/s41598-017-01087-7. The model parameters were estimated using a non-linear mixed effect approach using data collected in the COMPOSER study. Total Crovalimab, total C5, and 8 SEC fractions, where DTDCs are detected according to their molecular weight, were used to develop the model. The evaluation of model adequateness was satisfactory for simulation purposes. The model was calibrated using Eculizumab concentrations at the time of the switch and the time profiles of total Crovalimab, total C5 concentrations, and chromatography-based measurements of DTDC size distribution obtained from the Phase I/II COMPOSER study (see Roth et al., Blood (2020), Vol., 135, pp. 912-920; doi: 10.1182/blood 0.2019003399).

### 3.3 Phase III Dose Determination

The use of both models—the population pharmacokinetics model and the DTDC biochemical model—in parallel allowed the identification of a fixed-dose and dosing regimen that (1) minimizes the formation of larger DTDCs in patients switching from Eculizumab to Crovalimab, (2) maximizes the level of Crovalimab free binding sites, and (3) ensures that patients remain above the target threshold concentration required for complement inhibition (target C_{trough} above approximately 100 μg/mL Crovalimab) despite the inherent inter-individual variability.

Based on its mechanism of action, Crovalimab inhibits complement-mediated lysis of erythrocytes lacking complement regulatory proteins. If the terminal complement pathway is temporarily not blocked during the treatment interval, these erythrocytes will be lysed, and it may lead to breakthrough hemolysis, which is a severe clinical complication in PNH patients. Biological stress (infection, surgery, pregnancy) leads to a physiological activation of the complement pathway with upregulation of C5 (Schutte et al., Int Arch Allergy Appl Immunol (1975), Vol. 48(5), pp. 706-720.). In patients with PNH, it is therefore important to not only maintain complete blockade of the terminal complement activity throughout the dosing interval, but to also maintain a reserve of Crovalimab free binding sites to minimize the occurrence of breakthrough hemolysis.

Available pharmacokinetics (PK) and pharmacodynamics (PD) data from Parts 1, 2, and 3 from the COMPOSER study were integrated to enable characterization of the PK/PD relationship of Crovalimab following IV and SC administration and to identify the exposure levels required to completely inhibit the activity of the terminal complement system. By pooling the PK and PD data from the 9 healthy volunteers in Part 1, 10 patients with PNH in Part 2, and 16 patients with PNH in Part 3, Crovalimab was shown to induce a concentration-dependent inhibition of serum hemolytic activity, as measured by an ex vivo liposome immunoassay (LIA). Assessment of the exposure-response relationship demonstrates that approximately 100 μg/mL of Crovalimab is required to achieve complete terminal complement inhibition, defined as hemolytic activity <10 U/mL (see FIG. 1).

In the population PK model, body weight was tested as a covariate for Crovalimab clearance and volume of distribution and was found to statistically influence these parameters when incorporated using allometric scaling As a consequence, for a given dose, larger patients tend to have lower exposure be under-exposed as compared with smaller patients. To account compensate for the effect of body weight, a weight-based tiered dosing approach is proposed to ensure that all patients received a comparable Crovalimab exposure is achieved in all patients throughout the dosing interval.

The following two dosage regimens were determined:
For patients with a body weight >40 kg to <100 kg
Loading doses: Crovalimab 1000 mg intravenously administered (IV) on Day 1, followed by Crovalimab 340 mg subcutaneously (SC) administered on Days 2, 8, 15, and 22
Maintenance doses: Crovalimab 680 mg SC on Day 29, followed by subcutaneous administration of Crovalimab 680 mg SC once every 4 weeks (Q4W) thereafter.
For patients with a body weight >1=100 kg
Loading doses: Crovalimab 1500 mg IV on Day 1, followed by Crovalimab 340 mg SC on Days 2, 8, 15, and 22.
Maintenance doses: Crovalimab 1020 mg SC on Day 29, followed by subcutaneous administration of Crovalimab 680 mg SC once every 4 weeks (04W) thereafter.

### Example 4: Results of the DTDC Model Simulations

Simulations conducted from this model were aimed at identifying a dose and dosing regimen, minimizing the formation of larger DTDCs in patients switching from Eculizumab to Crovalimab, and providing sufficient free Crovalimab binding site reserves in patients switching from Eculizumab or treatment-naïve patients with PNH. The latter criterion provides an objective evaluation of the margin of hemolysis control that a dosing regimen provides to protect from breakthrough hemolysis. Simulations were performed only using parameter estimates from patients in COMPOSER Part 3 who switched from Eculizumab to Crovalimab. A dosing regimen providing a sufficient reserve of free Crovalimab epitopes in Eculizumab pre-treated patients is also appropriate for treatment of naïve patients. As shown in FIG. 2 and FIG. 3, the above mentioned dosing regimens are expected to maximize the availability of free epitopes while minimizing the formation of the largest DTDCs.

### Example 5: Results of the Population Pharmacokinetic Model Simulations

Simulations were conducted from the population PK model to recommend a dose and dosing regimen to ensure a rapid establishment of steady state concentrations as well as the maintenance of trough concentrations above 100 μg/mL in the majority of the patients throughout the dosing interval in both treatment-naïve and Eculizumab pre-treated PNH patients.

Crovalimab concentration—time profiles were simulated for 20,000 treatment-naïve patients with PNH and 20,000 patients with PNH who switched treatment from Eculizumab to Crovalimab with median body weight of 75.6 kg (standard deviation ±20.3 kg; with 42.2 kg and 109.0 kg the 5th and 95th percentiles, respectively). Simulations accounted for the age effect with 50% of the simulated population being above 50 years and with 50% of the simulated population being above 50 years. The choice of body weight distribution is based on the observed distribution in the COMPOSER study.

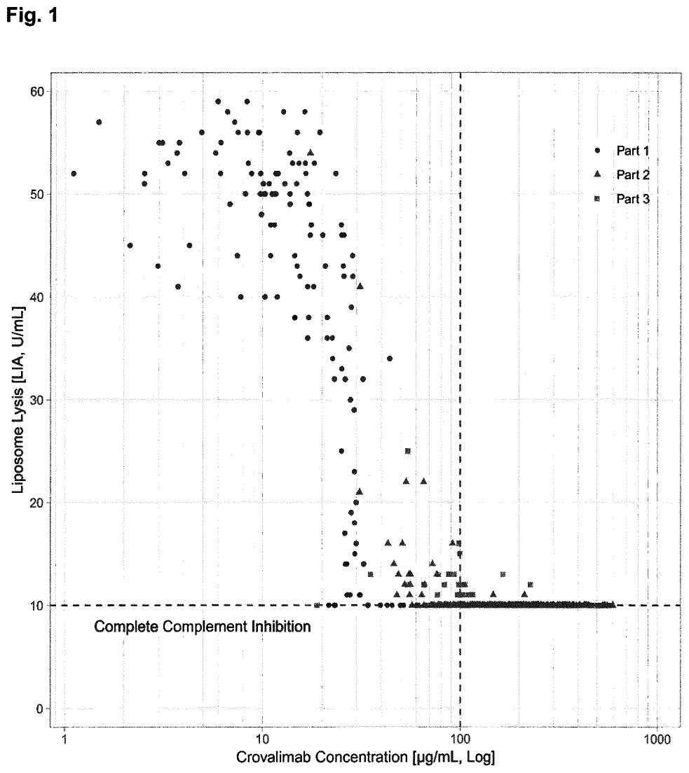

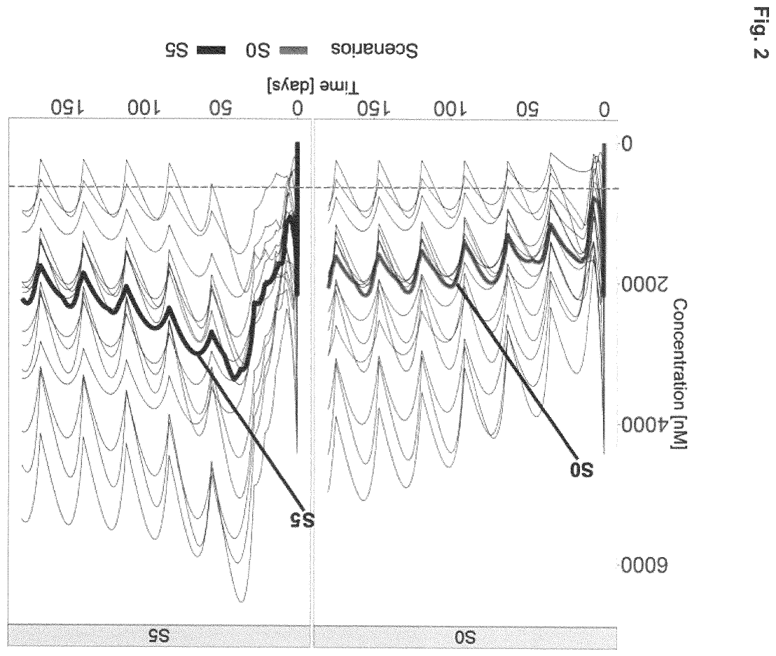

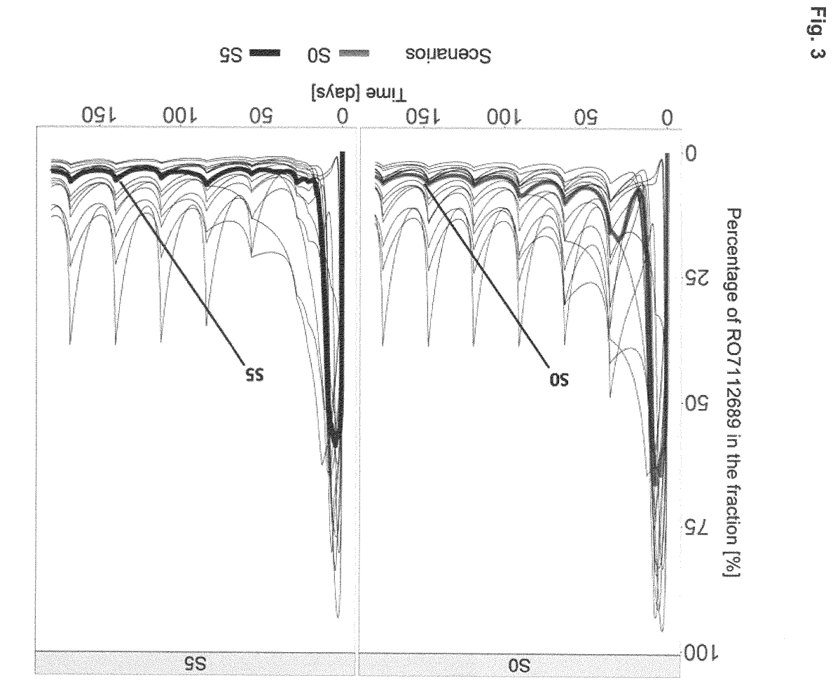

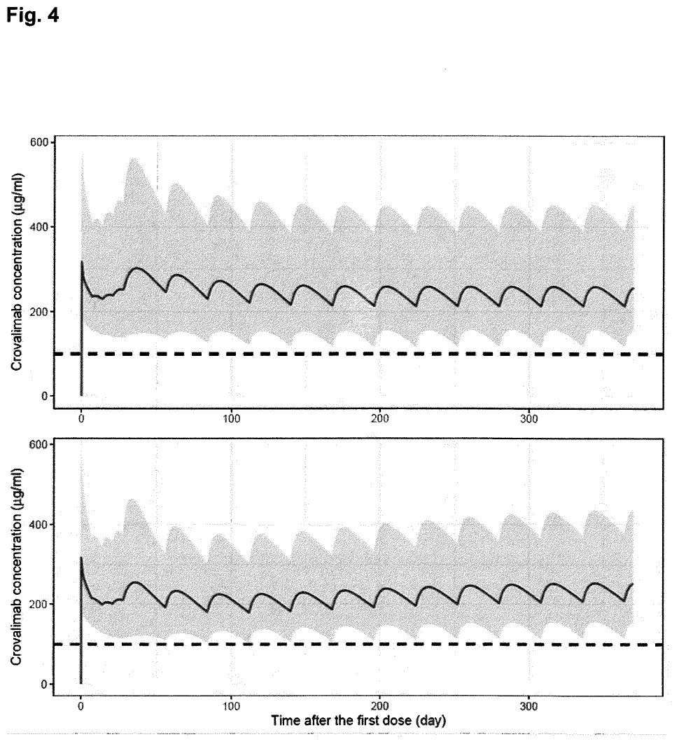

Based on the simulation results (FIG. 4), the above mentioned dosages and treatment regimen is predicted to result in rapid establishment of steady-state concentrations and sustained C_{trough} values greater than 100 μg/mL in approximately 95% of individuals throughout the dosing interval, regardless of body weight. This dosing regimen is predicted to maintain concentrations above 100 μg/mL in both treatment-naïve patients and patients switching from Eculizumab, despite the observed transient increase in Crovalimab clearance and the consequential longer time to reach steady-state concentrations in the latter.

The dose and dosing regimen proposed above is expected to ensure complete and consistent blockade of terminal complement activity (with approximately 95% of patients being maintained above the target threshold) and also ensure sufficient reserve of free binding sites for the majority of the dosing interval in both treatment-naïve and Eculizumab pre-treated patients. In patients switching from Eculizumab, it is also expected to reduce the formation of larger DTDCs. The above dosages were affirmed in Part 4 of the COMPOSER study in seven patients switching from Eculizumab to Crovalimab. Part 4 evaluated the safety, pharmacokinetics (PK) and pharmacodynamics (PD) effects of the above optimized Crovalimab regimen in 15 patients (data cut-off 29 Jan. 2020) with PNH who were naïve to the anti-C5 therapy (8 patients (53%)) or who had previously been treated with the anti-C5 antibody Eculizumab (7 patients (47%)). The baseline characteristics of patients enrolled in Part 4 of the COMPOSER study are shown in FIG. 6. The dosage most appropriate to reduce the persistence of DTDCs, particularly large DTDCs consisted of a loading dose series (Crovalimab 1000 mg intravenously administered (IV) on Day 1, followed by Crovalimab 340 mg subcutaneously (SC) administered on Days 2, 8, 15, and 22) followed by maintenance dosing (Crovalimab 680 mg SC on Day 29, followed by subcutaneous administration of Crovalimab 680 mg SC once every 4 weeks (Q4W) thereafter). The COMPOSER Part 4 data confirmed that the DTDC size distribution was shifted to smaller complexes with the claimed optimized dosing regimen. Further results for the above Crovalimab dose and regimen (Crovalimab 1000 mg intravenously administered (IV) on Day 1, followed by Crovalimab 340 mg subcutaneously (SC) administered on Days 2, 8, 15, and 22) followed by maintenance dosing (Crovalimab 680 mg SC on Day 29, followed by subcutaneous administration of Crovalimab 680 mg SC once every 4 weeks (Q4W) thereafter) reported in FIGS. 7 to 11.

As shown in FIG. 7, with this optimized dosage regimen, Crovalimab exposure was sustainably maintained above the C_{through} value of approximately 100 μg/mL (a level associated with complement inhibition) throughout a follow-up period of 20 Weeks (140 days).

Further, terminal complement inhibition was achieved immediately following the initial dose and maintained throughout the study period (see FIG. 8).

Further, a limited total C5 accumulation was observed in the PNH patients who were naïve to the anti-C5 therapy (8 patients; FIG. 9(A)) and a decline of the C5 levels was seen in the switched patients (PNH patients who had previously been treated with the anti-C5 antibody Eculizumab (7 patients; FIG. 9(B)). Further, FIG. 10 reports that the intravascular hemolysis was controlled and the majority of patients had hemoglobulin stabilisation and avoided blood transfusion: In total, 10 (67%) patients, including 5 of 8 naïve patients and 5 of 7 switched patients, achieved hemoglobin stabilsation (avoidance of ≥2 g/dL decrease in hemoglobin from baseline in the absence of blood transfusion) at Week 20. From baseline to Week 20, 11 (73%) patients, including 5 of 8 naïve patients and 6 of 7 switched patients, remained free of blood transfusion. Over 7.2 total patient years at risk, no patients experienced a breakthrough hemolysis (BTH) event as defined in Kulasekararaj et al., Blood (2019), Vol. 33, pp. 540-549.

Further, it was revealed that the above dose and treatment regimen of the anti-C5 antibody Crovalimab was well tolerated and no serious treatment-related adverse events (AEs) were observed (see FIG. 11).

Thus, the modelling approach described herein proves that the claimed dosage regimen is superior for the treatment or prevention of a C5-related disease such as PNH in both naïve and particularly Eculizumab pre-treated subjects.

### Example 6: Results of the Comparison of DTDC Size Distribution Between Part 3 and Part 4 of the COMPOSER Study

In COMPOSER Part 3, Drug-Target-Drug Complexes (DTDCs) between Crovalimab, human C5 and the antibody Eculizumab were detected in all patients with PNH who switched from the anti-C5 antibody Eculizumab to Crovalimab. The objective of the current example is to describe the results of the comparison of the DTDC size distribution between the dosage regimen of Part 3 and Part 4 of the COMPOSER study. In Part 3 of the COMPOSER study, the anti-C5 antibody Crovalimab is initially administered to the subjects intravenously once at the dose of 1000 mg/body. Starting from one week after the initial intravenous administration (day 8 after the IV administration), the anti-C5 antibody Crovalimab is subcutaneously (SC) administered once every week at the dose of 170 mg/body, once every two weeks at the dose of 340 mg/body, or once every four weeks at the dose of 680 mg/body. In Part 4 of the COMPOSER study the Crovalimab was administered according to the above dosage and treatment regimen: The optimized dose and regimen was a loading series of 1000 mg on day 1 and 340 mg SC on days 2, 8, 15, and 22, followed by maintenance dosing of 680 mg SC every 4 weeks starting on day 29 (week 5). The loading dose series increased the total dose of crovalimab received during the first month of treatment to reduce the formation of larger DTDCs, in line with the lattice theory of complex formation. This optimized dosing strategy was investigated in Part 4 patients who were switching treatments and compared with the 19 patients with PNH who enrolled in Part 3 and switched from Eculizumab to Crovalimab. DTDC size distributions were measured using size exclusion chromatography (SEC) coupled to ELISA. SEC separated the DTDC into fractions according to their size: Larger DTDCs are found in fractions 1-4 and smaller complexes, such as single motifs and non-DTDCs are found in fractions 5-6. DTDCs were observed in all patients from Part 3 (FIG. 12; larger DTDCs are found in fractions 1-4 and smaller complexes, such as single motifs and non-DTDCs are found in fractions 5-6). Two Part 3 patients experienced clinical manifestations compatible with type III hypersensitivity reactions that were ascribed to DTDCs. The DTDC size distribution in Part 4 patients, who received the optimized dosing strategy, evolved differently than in Part 3 patients, consistent with the model predictions. In the switched patients from Part 4 (n=7; data cut-off 29 Jan. 2020), the sum of DTDCs in fraction 1-4 started to decrease on Day 8 and continued to decrease, in contrast to Part 3. On Day 22, the mean percentage of the largest DTDCs was reduced by 56% in patients in Part 4 relative to patients in Part 3. Additionally, serum Crovalimab concentrations remained above 100 μg/mL for Part 4 patients, a level associated with complement inhibition. Despite DTDCs being observed in all Part 4 patients who switched from Eculizumab, no adverse events suggestive of a type III hypersensitivity reaction occurred. In conclusion, the optimized crovalimab regimen resulted in a lower concentration of large DTDCs than in patients who received the Part 3 regimen.

### Example 7: Results of the Response to Crovalimab of PNH Patients with C5 Polymorphism

Paroxysmal nocturnal hemoglobinuria (PNH) is characterized by the loss of endogenous complement regulators CD59 and CD55 on hematopoietic cells. Peripheral blood elements are susceptible to destruction by complement resulting in intravascular hemolysis and thrombosis. Standard therapy is terminal complement inhibition with Eculizumab, an anti-C5 monoclonal antibody (mAb). However, up to 3.5% of individuals of Asian descent carry polymorphisms in C5 affecting Arg885, which corresponds to the Eculizumab and Ravulizumab binding site (see Nishimura et al., N Engl J Med, Vol. 370, pp. 632-639 (2014); DOI: 10.1056/NEJMoa1311084). PNH patients with these polymorphisms experience poor control of intravascular hemolysis with Eculizumab, thus constituting a group with a high unmet medical need. Crovalimab is a novel anti-C5 mAb that binds a distinct epitope on the beta subunit of C5. In vitro studies have demonstrated that Crovalimab equally binds and inhibits the activity of wild-type and Arg885-mutant C5 (Fukuzawa et al., Sci Rep, 7(1): 1080. doi: 10.1038/s41598-017-01087-7 (2017)).

Objectives: The aim of the current example is to describe the response to Crovalimab of PNH patients with C5 polymorphism.

Methods: The above Crovalimab dose and regimen (Crovalimab 1000 mg intravenously administered (IV) on Day 1, followed by Crovalimab 340 mg subcutaneously (SC) administered on Days 2, 8, 15, and 22) followed by maintenance dosing (Crovalimab 680 mg SC on Day 29, followed by subcutaneous administration of Crovalimab 680 mg SC once every 4 weeks (Q4W) thereafter) were administered to PNH patients with C5 polymorphism (Arg885 mutation of C5 (SEQ ID NO: 13)). Plasma concentration of Crovalimab, lactate dehydrogenase (LDH), free and total C5, and complement activity were determined at every visit. Patients were followed for occurrence of blood transfusions, breakthrough hemolytic (BTH) events, and for safety.

Results: Of the 44 patients enrolled in part 2 (n=10), part 3 (n=19) and part 4 (n=15) of the COMPOSER study (ClinicalTrials.gov Identifier: NCT03157635), four had the c.2654G->A nucleotide polymorphism predicting Arg885His substitution. At the September 2019 data cut-off, follow-up ranged from 12.4-98.3 weeks. All four patients were male, diagnosed 44-734 weeks before enrollment with PNH granulocyte clone size ranging from 89-95%. At enrollment, one patient switched from ongoing therapy with Eculizumab while three had previously discontinued Eculizumab. All patients had LDH>3-fold upper limit of normal (ULN) at enrollment which declined rapidly and was maintained at less than 1.5×ULN throughout the follow-up period (FIG. 13). One patient required transfusions after enrollment (12 units of red blood cells (RBC) over 6 months); this patient had an underlying diagnosis of aplastic anemia and required 198 units of RBC in the 12 months prior to enrollment. None of the four patients experienced a breakthrough hemolytic (BTH) event. All four patients achieved complete terminal complement inhibition as measured by liposome immunoassay (LIA). LIA levels ranged from 32-42 U/mL at study entry and declined to <10 U/mL (lower level of quantification) by day 2 and were maintained thereafter. Similarly, free C5 levels were maintained at <0.5 μg/mL after week 6 (day 43). The safety profile of these patients was similar to the remainder of the participants. Three serious adverse events (SAEs) were reported, none of which were related to study treatment. One patient had two SAEs, bile duct stone and cholelithiasis. A second patient had an SAE of upper respiratory tract infection with admission to the hospital, which occurred after 20 months and resolved while on treatment.

Conclusions: Crovalimab achieved complete and sustained terminal complement inhibition in PNH patients with Arg885 polymorphism. Thus, Crovalimab is a promising anti-C5 antibody for the treatment and/or prevention of patients suffering from PNH, wherein the patients are characterized by having the C5 Arg885His mutation.

1. A method of treating or preventing a C5-related disease in a subject, wherein the method comprises the consecutive steps of:
(a) intravenously administering a loading dose of 1500 mg of anti-C5 antibody to the subject once, followed by subcutaneously administering at least one loading dose of 340 mg of the anti-C5 antibody to the subject; and
(b) subcutaneously administering at least one maintenance dose of 1020 mg of the anti-C5 antibody to the subject.

2. The method according to claim 1, wherein the subcutaneously administered loading dose of 340 mg of the anti-C5 antibody is administered at least once to the subject 1 day to 3 weeks after the start of the intravenous administration of the anti-C5 antibody.

3. The method according to claim 2, wherein the subcutaneously administered loading dose of 340 mg of the anti-C5 antibody is administered once to the subject 1 day after the start of the intravenous administration of the anti-C5 antibody.

4. The method according to claim 2, wherein at least one additional loading dose of 340 mg of the anti-C5 antibody is subcutaneously administered to the subject 1 week or 2 weeks after the start of the intravenous administration of the anti-C5 antibody.

5. The method according to claim 2, wherein an additional loading dose of 340 mg of the anti-C5 antibody is subcutaneously administered to the subject 1 week and 2 weeks after the start of the intravenous administration of the anti-C5 antibody once weekly.

6. The method according to claim 1, wherein at least one maintenance dose of 1020 mg of the anti-C5 antibody is subcutaneously administered to the subject 4 weeks after the start of the intravenous administration of the anti-C5 antibody.

7. The method according to claim 6, wherein the maintenance dose of 1020 mg of the anti-C5 antibody is subcutaneously administered once to the subject 4 weeks after the start of the intravenous administration of the anti-C5 antibody.

8. The method according to claim 6, wherein the subcutaneous administration of a maintenance dose of 1020 mg of the anti-C5 antibody to the subject is repeated several times with time intervals of at least 4 weeks.

9. The method according to claim 1, wherein the method is comprises
(i) intravenously administering a loading dose of 1500 mg of the anti-C5 antibody to the subject once;
(ii) subcutaneously administering a loading dose of 340 mg of the anti-C5 antibody to the subject 1 day after the start of the intravenous administration of the anti-C5 antibody;
(iii) subcutaneously administering a loading dose of 340 mg of the anti-C5 antibody to the subject 1 week, 2 weeks and 3 weeks after the start of the intravenous administration of the anti-C5 antibody once weekly;
(iv) subcutaneously administering a maintenance of 1020 mg of the anti-C5 antibody to the subject 4 weeks after the start of the intravenous administration of the anti-C5 antibody; and
(v) repeating step (iv) several times with time intervals of 4 weeks.

10. The method according to claim 1, wherein the subject received prior treatment with at least one pharmacological product useful for the treatment or prevention of the C5-related disease, wherein the intravenously administered loading dose of 1500 mg of the anti-C5 antibody is administered to the subject after the final dose of the pharmacological product.

11. The method according to claim 10, wherein the intravenously administered loading dose of 1500 mg of the anti-C5 antibody is administered to the subject on the third day or after 3 days after administration of the final dose of the pharmacological product.

12. The method according to claim 10, wherein the pharmacological product comprises an siRNA targeting C5 mRNA, or an anti-C5 antibody which is different from the anti-C5 antibody in the loading dose and the maintenance dose.

13. The method according to claim 10, wherein the pharmacological product comprises Eculizumab, Ravulizumab or variants thereof.

14. The method according to claim 1, wherein the subject has a body weight of equal or greater than 100 kg.

15. The method according to claim 1, wherein the anti-C5 antibody concentration determined in a biological sample of said subject is 100 μg/ml or more.

16. The method according to claim 1, wherein a hemolytic activity determined in a biological sample of said subject is less than 10 U/mL.

17. The method according to claim 15, wherein the biological sample is a blood sample, preferably a red-blood sample.

18. The method according to claim 1, wherein the anti-C5 antibody is Crovalimab.

19. The method according to claim 1, wherein the C5-related disease is selected from a group consisting of paroxysmal nocturnal hemoglobinuria (PNH), rheumatoid arthritis (RA), lupus nephritis, ischemia-reperfusion injury, atypical hemolytic uremic syndrome (aHUS), dense deposit disease (DDD), macular degeneration, hemolysis, elevated liver enzymes, low platelets (HELLP) syndrome, thrombotic thrombocytopenic purpura (TTP), spontaneous fetal loss, Pauci-immune vasculitis, epidermolysis bullosa, recurrent fetal loss, multiple sclerosis (MS), traumatic brain injury, an injury resulting from myocardial infarction, cardiopulmonary bypass or hemodialysis, refractory generalized myasthenia gravis (gMG), and neuromyelitis optica (NMO).
